(19) United States
(12) Patent Application Publication
(10) Pub. No.: US 2022/0010019 A1
(43) Pub. Date: Jan. 13, 2022

(54) Targeting intraepithelial lymphocytes for treatment of metabolic syndrome

(71) Applicant: The General Hospital Corporation, Boston, MA (US)

(72) Inventors: Filip K. Swirski, Swampscott, MA (US); Shun He, Malden, MA (US)

(21) Appl. No.: 17/297,153

(22) PCT Filed: Nov. 26, 2019
(86) PCT No.: PCT/US19/63253

§ 371 Date: May 26, 2021

Related U.S. Application Data
(60) Provisional application No. 62/771,668, filed on Nov. 27, 2018.
(60) Provisional application No. 62/923,133, filed on Oct. 18, 2019.

(51) Int. Cl. C07K 16/28; A61K 38/26

(52) U.S. Cl. CPC C07K 16/2839; A61K 2039/505; A61K 38/26

(57) ABSTRACT

Methods and compositions of treating patients with metabolic syndrome or a disease associated with metabolic syndrome using inhibitors that target natural intraepithelial lymphocytes.

### CLAIM OF PRIORITY

This application claims the benefit of U.S. Provisional Patent Application Ser. No. 62/771,668, filed on Nov. 27, 2018; and Ser. No. 62/923,133, filed on Oct. 18, 2019. The entire contents of the foregoing are hereby incorporated by reference.

### FEDERALLY SPONSORED RESEARCH OR DEVELOPMENT

This invention was made with Government support under Grant Nos. HL135752, HL128264, and HL131478 awarded by the National Institutes of Health. The Government has certain rights in the invention.

### TECHNICAL FIELD

The invention relates to methods of treating and/or preventing metabolic syndrome by targeting intraepithelial lymphocytes.

### BACKGROUND

“Metabolic syndrome” refers to the co-occurrence of several known cardiovascular risk factors, including insulin resistance, obesity, diabetes, hypertension, hypercholesterolemia, and atherogenic dyslipidemia. Metabolic syndrome is a disorder of energy intake, utilization and storage, diagnosed by a co-occurrence of three out of five of the following medical conditions: abdominal obesity, elevated blood pressure, elevated fasting plasma glucose, high serum triglycerides, and low high-density cholesterol (HDL) levels. The metabolic syndrome is thus a combination of metabolic disorders, resulting in hyperlipidemia, impaired glucose tolerance, hypertension, oxidative stress and the tendency to develop fat around the abdomen. Individuals with metabolic syndrome are at high risk of developing heart failure and insulin resistance, thus affecting vital organs such as the eye, liver, kidney and nervous system. Although various risk factors such as age, high body mass index, smoking, stress, sedentary lifestyle, and postmenopausal status are identified, high fat diet is one of the most important risk factors leading to metabolic syndrome. Chronic metabolic syndrome has become a prominent public health concern. While medical treatment has been effective in the treatment of certain metabolic syndrome related diseases (like in diabetes mellitus), the incidence of these disorders continues to be high.

The immune system protects us against pathogens such as viruses, bacteria, and parasitic worms, but its influence is much broader. The system recognizes and responds to divergent environmental and endogenous stimuli, and every known disease is at least partially associated with or dependent on immune function. Cardiovascular disease is no exception.

Atherosclerosis is the pathology that leads to myocardial infarction and stroke. For many years after its recognition, atherosclerosis was thought to involve passive lipid deposition in the vessel wall. Today we understand that atherosclerosis is a chronic inflammatory disease driven by lipids, specifically low density lipoproteins (LDL) and leukocytes. Neither atherosclerosis nor its complications adhere to a simple arithmetic of dietary lipid imbalance, but rather comprise a syndrome in which environmental and genetic inputs disrupt biological systems. In other words, lifestyle, age, hereditary factors, and co-morbidities disturb immune, digestive, endocrine, circulatory, and nervous systems, thereby altering immune function, metabolism, and many other processes, while eliciting inflammation, hypercholesterolemia, and hypertension. Atherosclerosis develops and causes myocardial infarction or stroke when many things go wrong in many different ways.

### SUMMARY

Provided herein are methods of reducing risk of developing metabolic syndrome or a disease associated with metabolic syndrome in a subject with a family history of metabolic syndrome or a disease associated with metabolic syndrome, elevated blood pressure, dysglycemia, or abdominal obesity, the method comprising administering a therapeutically effective amount of a β7 integrin inhibitor to the subject in need thereof.

Also provided herein are methods of treating a subject who has been diagnosed with metabolic syndrome or a disease associated with metabolic syndrome, the method comprising administering a therapeutically effective amount of a β7 integrin inhibitor to the subject in need thereof.

Also provided herein are methods of reducing risk of developing metabolic syndrome or a disease associated with metabolic syndrome in a subject with family history of metabolic syndrome or a disease associated with metabolic syndrome, elevated blood pressure, dysglycemia, or abdominal obesity, the method comprising administering a therapeutically effective amount of a CCR9 inhibitor or a GLP-1 agonist to the subject in need thereof.

In some embodiments, the metabolic syndrome or the disease associated with metabolic syndrome is atherosclerosis.

In some embodiments, the β7 integrin inhibitor is natalizumab or vedolizumab or etrolizumab.

In some embodiments, the therapeutically effective amount of the β7 integrin inhibitor is sufficient to inhibit intraepithelial lymphocyte recruitment to small intestine.

In some embodiments, the β7 integrin inhibitor is delivered directly to the small intestine of the subject, e.g., intraperitoneally, subcutaneously, or by administering the β7 integrin inhibitor in an oral form that remains intact in the stomach but releases the inhibitor once in the small intestine.

In some embodiments, the subject has not been diagnosed with a metabolic syndrome or the disease associated with metabolic syndrome.

In some embodiments, the subject does not have a chronic inflammatory bowel disease.

In some embodiments, the chronic inflammatory bowel disease is irritable bowel syndrome, Crohn's disease, or ulcerative colitis.

In some embodiments, the CCR9 inhibitor is CCX025 or CCX282.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-1L show integrin β7 regulates metabolism. A-D, Body weight (A), cumulative food intake (B), energy expenditure (C) and heat production (D) in wild-type (β7+/+) and β7−/− mice fed a chow diet. n=5 mice per group. E, Representative (of 6 and 5 mice, respectively) PET/CT images after [¹⁸F]FDG administration to wild-type and β7−/− mice. F, Standard update values (SUV) quantified in vivo in indicated regions of interest. n=6 wild-type mice; n=5 β7−/− mice. A nonparametric multiple comparisons test was used. BAT, brown adipose tissue; iWAT, inguinal white adipose tissue. G, Left, glucose-tolerance test in wild-type and β7−/− mice that were fed a chow diet; after intraperitoneal glucose injection. Right, area under curve (AUC) of intraperitoneal glucose-tolerance test. n=17 wild-type mice; n=16 β7−/− mice. H, Plasma insulin levels in wildtype and β7−/− mice 15 min after glucose stimulation. n=4 wild-type mice; n=5 β7−/− mice. I, Insulin-tolerance test in wild-type and β7−/− mice fed a chow diet. n=5 wild-type mice; n=4 β7 / mice. J, Plasma triglyceride (TG) levels of fasted wild-type and β7−/− mice. n=31 wildtype mice; n=27 β7−/− mice. K, Fat-tolerance test in wild-type and β7−/− mice fed a chow diet after intraperitoneal injection of 20% intralipid. n=5 mice per group; ***P<0.001, two-way analysis of variance (ANOVA). L, Hepatic triglyceride secretion. Overnight-fasted wild-type and β7−/− mice were injected intraperitoneally with the lipase inhibitor poloxamer 407 (P407) and plasma triglyceride levels were determined at the indicated time points. n=4 wild-type mice; n=3 β7−/− mice. Data are mean±s.e.m., representing biological replicates. *P<0.05, **P<0.01, ***P<0.001, ****P<0.0001, two-tailed Mann-Whitney U-tests unless otherwise indicated.

FIGS. 2A-2G show integrin β7 deficiency protects against metabolic syndrome. A, Body weights of wild-type and β7−/− mice that were fed a HFSSD for 5 months. n=9 wild-type mice; n=8 β7−/− mice. Representative images of wild-type and β7−/− mice are shown on the left. Black dots denote animals shown in the images. B, Tissue weights of wild-type and β7−/− mice after 5 months of a HFSSD. n=10 mice per group, except for heart, n=5. C, Representative haematoxylin and eosin images of iWAT and pWAT of wild-type (n=5) and β7−/− (n=4) mice fed a HFSSD for 5 months. D, E, Quantification of adipocytes at indicated size ranges in iWAT and pWAT of wild-type and β7−/− mice fed a HFSSD for 5 months. n=5 wild-type mice; n=4 β7−/− mice. A nonparametric multiple-comparisons test was used. F, Blood pressure measurements of mice fed a HFSSD. n=5 mice per group. G, Left, glucose-tolerance test using oral glucose gavage (2 g per kg body weight) in wild-type and β7−/− mice fed a HFSSD for 5 months. Right, AUC of the glucose-tolerance test. n=10 wild-type mice; n=7 β7−/− mice. Data are mean±s.e.m., representing biological replicates. *P<0.05, **P<0.01, ***P<0.001, ****P<0.0001, two-tailed Mann-Whitney U-tests unless otherwise indicated.

FIGS. 3A-3G show integrin β7 deficiency protects against atherosclerosis. Ldlr−/− mice were lethally irradiated and reconstituted with bone marrow cells from either wild-type (bmβ7+/+Ldlr−/−) or β7−/− (bmβ7−/−Ldlr−/−) mice. A, Plasma cholesterol in fed and overnight-fasted animals fed a HCD for 14 weeks. n=6 bmβ7+/+Ldlr−/− mice; n=9 bmβ7−/−Ldlr−/− fed mice; n=10 bmβ7+/+Ldlr−/− mice; n=9 bmβ7−/−Ldlr−/− fasted mice. B, Plasma lipoprotein distribution measured by fast-performed liquid chromatography in bmβ7+/+Ldlr−/− and bmβ7−/−Ldlr−/− mice. HDL, high-density lipoprotein; IDL, intermediate-density lipoprotein; LDL, low-density lipoprotein; VLDL, very-low-density lipoprotein. Plasma from n=5 mice per group was pooled. c, d, Representative images (C) and quantification (D) of oil-red O staining of aortic root sections from bmβ7+/+Ldlr−/− and bmβ7−/−Ldlr−/− mice fed a HCD for 14 weeks. n=12 bmβ7+/+Ldlr−/− mice; n=13 bmβ7−/−Ldlr−/− mice. E, Plaque volumes were calculated by measuring the plaque size at increasing distances from the aortic root. n=5 mice per group; *P<0.05, two-tailed unpaired Student's t-test. F, G, Leukocyte quantification in aortas (F) and blood (G) of bmβ7+/+Ldlr−/− and bmβ7−/−Ldlr−/− mice fed a HCD for 14 weeks. n=6 bmβ7+/+Ldlr−/− and n=5 bmβ7−/−Ldlr−/− in F; n=5 mice per group in G Data are mean±s.e.m., representing biological replicates. *P<0.05, **P<0.01, ***P<0.001, two-tailed Mann-Whitney U-tests unless otherwise indicated.

FIGS. 4A-4N show natural IELs calibrate metabolism and protect against cardiovascular disease through GLP-1. A, Histogram (of n=4) showing integrin β7 expression on leukocytes in wild-type mice. BM, bone marrow; iso, isotype control; SI-IELs, small-intestine intraepithelial leukocytes. B, Flow cytometry plots showing leukocyte chimerism in blood and among SI-IELs. CD45.2+ mice were lethally irradiated and transplanted with a 1:1 ratio mixture of GFP+β7−/− and CD45.1+ wild-type bone marrow cells. C, T cell quantification in small intestine in the same mice as b. n=4 recipient mice. D, Wild-type mice were lethally irradiated and reconstituted with bone marrow cell mixtures to generate mixed chimeric mice. Data show glucose-tolerance tests (GTT) and the AUC in β7−/− wild-type (WT) (n=4 mice), β7−/−βTCR−/− (n=3 mice), and β7−/−γδ TCR−/− (n=5 mice) mice. KO indicates knockout (either βTCR−/− or γδ TCR−/−) bone marrow. E, Glucose tolerance test (intraperitoneal injection) in wild-type (n=4 and 5 mice) and Itgae−/− (n=4 mice) or Ccr9−/− mice (n=3 mice). F, G, Fasting plasma total GLP-1 (tGLP-1) levels (F) and Gcg mRNA expression (G) in the ileum of bmβ7+/+Ldlr−/− (n=5 mice) and bmβ7−/−Ldlr−/− (n=4 and 5 mice) mice fed a HCD for 14 weeks. H, Glp1r in sorted IELs from wild-type mice. n=3 mice. Epi., epithelial cells; Mye., myeloid cells; Nat., natural; Ind., induced. I, Experimental set-up for generating mixed bone marrow chimaeras. J, Fasting plasma total GLP-1 levels. n=6 bmGlp1r+/+β7−/−Ldlr−/− mice; n=4 bmGlp1r−/−β7−/−Ldlr−/− mice. K, Glucose-tolerance test. n=5 mice. L, Plasma cholesterol. n=5 bmGlp1r+/+β7−/−Ldlr−/− mice; n=4 bmGlp1r−/−β7−/−Ldlr−/− mice. *P<0.05, two-tailed unpaired Student's t-test. M, Representative images and quantification of oil-red O-stained aortic roots. n=4 bmGlp1r+/+β7−/−Ldlr−/− mice; n=6 bmGlp1r−/−β7−/−Ldlr−/− mice. N, Quantification of the number of leukocytes in aortas. n=5 mice. K-N, Mice were fed a HCD. Data are mean±s.e.m., representing biological replicates. *P<0.05, **P<0.01, two-tailed Mann-Whitney U-tests unless otherwise indicated.

FIGS. 5A-5I show effect of integrin β7 deficiency on metabolism. A, Metabolic cage measurements of activity in wild-type (WT) and β7−/− mice. n=4 mice per group. B, O2 consumption and CO2 production. n=5 wild-type mice; n=4 β7−/− mice. C, Respiratory exchange rate (RER) by CLAMS in wild-type and β7−/− mice that were fed a chow diet. n=5 wild-type mice; n=4 β7−/− mice; *P<0.05, two-tailed Mann-Whitney U-test. D, Overnight-fasted wild-type and β7−/− mice were administered [¹⁸F]FDG. The radioactivity in indicated organs was measured as the percentage injected dose per gram tissue weight (% ID/g). n=6 wild-type mice; n=5 β7−/− mice; *P<0.05, Student's t-test. E, Wild-type and β7−/− mice were housed in thermoneutral (TN) incubators for three days and then subjected to the intraperitoneal (IP) glucose-tolerance test. n=5 mice per group; **P<0.01, two-tailed Mann-Whitney U-test. F, Wild-type and β7−/− mice were treated with antibiotic cocktails in drinking water for 4 weeks and then subjected to the glucose-tolerance test. n=4 mice per group; *P<0.05, two-tailed Mann-Whitney U-test. G, Eight-week-old wild-type and β7−/− mice were cohoused at a ratio of 1:1 for 4 weeks and then subjected to the glucose-tolerance test. n=7 mice per group; *P<0.05, two-tailed Mann-Whitney U-test. H, Fat absorption was analysed using a fat-tolerance test in the presence of P407. n=9 wild-type mice; n=6 β7−/− mice. I, For assessment of permeability, mice were gavaged with FITC-dextran and fluorescence was measured in the plasma 4 h later. A wild-type mouse subjected to a dextran sulfate sodium (DSS)-induced colitis model was used as a positive control for increased gut permeability. n=8 wild-type mice; n=7 β7−/− mice; P=0.17, two-tailed Mann-Whitney U-test. Data are mean±s.e.m.

FIGS. 6A-6J show effect of integrin β7 deficiency on obesity, cholesterolaemia and atherosclerosis. A, Representative flow cytometry plots gated on CD45+ non-T and B (TB) leukocytes and quantification of Ly-6Chigh monocytes, neutrophils and macrophages (Mφ) in iWAT of wild-type and β7−/− mice fed a HFSSD for 5 months. n=5 wild-type mice; n=4 β7−/− mice; *P<0.05, two-tailed Mann-Whitney U-test. B, Representative flow cytometry plots and quantification of Ly-6Chigh monocytes, neutrophils and macrophages in pWAT of wild-type and β7−/− mice fed a HFSSD for 5 months. n=5 wild-type mice; n=4 β7−/− mice; *P<0.05, two-tailed Mann-Whitney U- test. C, Plasma glucose levels measured in overnight-fasted animals fed a HFSSD for 5 months. n=10 wild-type mice; n=7 β7−/− mice; ***P<0.001, two-tailed Mann Whitney U-test. D, Insulin levels measured in overnight-fasted animals fed a HFSSD for 5 months. n=9 wild-type mice; n=8 β7−/− mice; *P<0.05, two-tailed Mann-Whitney U-test. E, Fasting plasma total cholesterol levels of mice fed a chow diet. n=6 mice per group. F, Body weight changes during a 14-week HCD diet of bmβ7+/+Ldlr−/− and bmβ7−/−Ldlr−/− mice. n=5 mice per group. G, Faecal cholesterol levels in bmβ7+/+Ldlr−/− and bmβ7−/−Ldlr−/− mice after a 14-week HCD diet. n=5 mice per group; P=0.09, two-tailed unpaired Student's t-test. H-J, Representative images and histological quantification of macrophages (H), collagen content and necrotic core size (I) and smooth muscle cell content (J) of bmβ7+/+Ldlr−/− and bmβ7−/−Ldlr−/− mice after 14 weeks on a HCD. n=5 mice per group; **P<0.01, ***P<0.001, two-tailed unpaired Student's t-test. Data are mean±s.e.m.

FIGS. 7A-7D show effect of integrin β7 deficiency on myeloid cells and glucose tolerance. A, Ldlr−/− mice were lethally irradiated and reconstituted with bone marrow mixtures of wild-type and β7−/− mice (1:1) and fed a chow diet or HCD for 14 weeks. B, The aortic leukocytes from different origins were analysed by flow cytometry. n=4 mice for both HCD recipients and chow recipients. C, Ly-6Chigh and Ly-6Clow monocyte numbers in blood (n=3 wild-type mice; n=5 β7−/− mice), bone marrow (n=3 wild-type mice; n=5 β7−/− mice) and spleen (n=6 mice per group) of wild-type and β7−/− mice fed a chow diet. D, bmβ7+/+Ldlr−/− and bmβ7−/−Ldlr−/− mice fed a HCD were subjected to an intraperitoneal glucose-tolerance test. n=5 mice per group; *P<0.05, two-tailed Mann-Whitney U-test. Data are mean±s.e.m.

FIGS. 8A-8F show effects of genetic deficiency and blocking of integrin β7 on atherosclerosis. A, Body weights, cumulative food intake and energy expenditure were measured in Ldlr−/− mice and β7−/−Ldlr−/− mice. n=4 mice per group. B, Ldlr−/− mice and β7−/−Ldlr−/− mice were fed a HCD for 14 weeks. Plasma cholesterol levels were determined in overnight-fasted mice. n=7 Ldlr−/− mice; n=5 β7−/−Ldlr−/− mice; **P<0.01, two-tailed Mann-Whitney U-test. C, Representative oil-red O images and quantification of plaque size in the aortic roots. n=7 Ldlr−/− mice; n=5 β7−/−Ldlr−/− mice; *P<0.05, two-tailed Mann-Whitney U-test. D, Quantification of Ly-6Chigh monocytes, neutrophils and macrophages in plaques. n=7 mice per group. *P<0.05, **P<0.01, two-tailed Mann-Whitney U-test. E, Ldlr−/− mice that were fed a HCD were treated with anti-integrin β7 antibodies or IgG isotype control (500 μg per mouse per week) for 14 weeks. Mice were subjected to a glucose-tolerance test after 8 weeks on the HCD. n=6 mice per group; **P<0.01, two-tailed Mann-Whitney U-test. F, Representative images of oil-red O-stained aortic cross-sections and quantification of plaque size in the aortic roots after 14 weeks on a HCD. n=5 mice treated with IgG; n=6 mice treated with anti-integrin β7 antibody; *P<0.05, **P<0.01, two-tailed Mann-Whitney U-test. Data are mean±s.e.m.

FIGS. 9A-9F show integrin β7 guides leukocytes to gut intraepithelium. A, Top, representative histology staining for CD3 in small intestines of wild-type and β7−/− mice. Bottom, quantification of CD3+ cells in each villus (more than 15 villi were counted for each mouse). ***P<0.001, two-way ANOVA. B, Schematic of the competitive transfer experiments. Mice (CD45.2+) were lethally irradiated and transplanted with a 1:1 ratio mix of GFP+β7−/− bone marrow cells and CD45.1+ wild-type bone marrow cells. The chimerism in different tissues was normalized to the ratio in blood. n=5 mice. C, Representative flow cytometry plots and quantification of B cells and myeloid cells in mice depicted in B. n=5 biologically independent recipients. D, Quantification of γδ T cells in the liver. n=3 wild-type mice; n=4 β7−/− mice. WBC, white blood cells. E, Quantification of γδT cells in the pancreas. n=4 wild-type mice; n=5 β7−/− mice. F, Wild-type mice were lethally irradiated and reconstituted with bone marrow cell mixtures of β7−/− and wild-type (β7−/−:wild-type cells, 1:1 ratio) or β7−/− and indicated knockout mice (β7−/−:knockout cells, 1:1 ratio). The indicated mixed chimaeras that specifically lack intestinal B cells (β7−/−μMT) or myeloid cells (β7−/−Ccr2−/−) were subjected to oral glucose-tolerance tests and the AUCs are shown. n=5 mice per group for β7−/− wild-type and β7−/−μMT mice; n=4 mice per group for β7−/− wild-type and β7−/−Ccr2−/− mice. Data are mean±s.e.m.

FIGS. 10A-10E show B cells are dispensable for the altered metabolic phenotypes in integrin β7-deficient mice. Ldlr−/− mice were lethally irradiated and reconstituted with bone marrow cell mixtures of β7−/− and wild-type (β7−/− WT, 1:1 ratio) or β7−/− and μMT (β7−/−μMT, 1:1 ratio). The reconstituted mixed chimaeras were fed a HCD for 14 weeks. A, IgA levels in the gut flush (n=5 β7−/− wild-type mice; n=4 β7−/−μMT) and plasma (n=5 β7−/− wild-type mice; n=3 β7−/−μMT mice). **P<0.01, two-tailed Mann-Whitney U-test. B, Number of IgD+ B cells in Peyer's patches (PP) and IgA+ B cells and IgD+ B cells in lamina propria (LP) as determined by flow cytometry. n=5 β7−/− wild-type mice; n=3 β7−/−μMT mice; *P<0.05, two-tailed Mann-Whitney Utest. C, Glucose-tolerance test in HCD-fed mixed chimaeras. n=5 β7−/− wild-type mice; n=3 β7−/−μMT mice. D, Plasma cholesterol levels in overnight-fasted mice. n=5 β7−/− wild-type mice; n=4 β7−/−μMT mice. E, Representative images and quantification of oil-red O staining in aorta root sections of bmβ7−/− wild-type Ldlr−/− mice and bmβ7−/−μMT Ldlr−/− mice that were fed a HCD for 14 weeks. n=5 bmβ7−/− wild-type Ldlr−/− mice; n=4 bmβ7−/−μMT Ldlr−/− mice. Data are mean±s.e.m.

FIGS. 11A-11E show integrin β7 deficiency and GLP-1. A, Plasma total GLP-1 levels after overnight fasting and 15 min after oral glucose load (2 g per kg body weight) in wild-type and β7−/− mice that were fed a chow diet. Total GLP-1 fasting: n=7 mice per group; total GLP-1 after oral glucose tolerance test (OGTT) 15 min: n=7 wild-type mice; n=6 β7−/− mice. B, Plasma total GLP-1 levels after 5 months of a HFSSD. Total GLP-1 fasting: n=7 mice per group; total GLP-1 oral glucose-tolerance test 15 min: 6 mice per group. C, Representative flow cytometry plots of small-intestinal IELs from wild-type and β7−/− mice. D, Glp1r mRNA levels in sorted different IEL subsets from wild-type and β7−/− mice. n=4 wildtype mice; n=5 β7−/− mice. E, Wild-type mice were lethally irradiated and transplanted with a 1:1 bone marrow mixture of wild-type and GFP+ or Glp1r−/− and GFP+. The chimerism in different tissues was analysed by comparing the percentage of GFP+ leukocytes normalized to wild-type GFP+ blood leukocytes. n=4 mice per group. Data are mean±s.e.m. *P<0.05, ***P<0.001. All P values from two-tailed unpaired Student's t-tests.

FIGS. 12A-12K show effect of Glp1r deficiency on IELs and atherosclerosis. A, Quantification of small-intestinal IEL subpopulations in bmGlp1r+/+β7−/−Ldlr−/− and bmGlp1r−/−/β7−/−Ldlr−/− mice. n=5 mice per group, mean±s.e.m. B, Glp1r mRNA expression of sorted IEL subpopulations. n=4 biologically independent bmGlp1r+/+β7−/− and n=5 biologically independent bmGlp1r−/−β7−/− mice; two-tailed unpaired Student's t-test. C, Glp1r mRNA expression in the liver (n=5 bmGlp1r+/+β7−/− and n=4 bmGlp1r−/−β7−/− mice), heart and lung tissue (n=5 mice per group). D, Quantification of γδ T cells from the liver (n=5 mice per group) and pancreas (n=5 bmGlp1r+/+β7−/−Ldlr−/− mice; n=4 bmGlp1r−/−β7−/−Ldlr−/− mice). E, Glp1r mRNA expression of sorted γδ T cells from pancreas, liver and small-intestinal IELs. n=3 mice per group; two-tailed unpaired Student's t-test. F, Oral glucose tolerance test. n=4 bmGlp1r+/+ mice; n=3 bmGlp1r−/− mice. G, GLP-1 levels after overnight fasting (n=4 mice per group) or oral glucose challenge (n=4 bmGlp1r+/+ mice; n=3 bmGlp1r−/− mice). H, Oral glucose-tolerance test in bmGlp1r+/+β7−/− and bmGlp1r−/−β7−/− mice. n=5 mice per group; two-tailed Mann-Whitney U-test. I, GLP-1 levels after overnight fasting or oral glucose challenge in bmGlp1r+/+β7−/− and bmGlp1r−/−β7−/− mice. n=5 mice per group; two-tailed unpaired Student's t-test. J, Ldlr−/− mice were treated with the GLP-1 receptor agonist exendin-4 (Ex-4) at a dose of 100 μg per kg per day via osmotic minipumps (PBS was used as control). After 8 weeks on a HCD, mice were euthanized to enable the quantification of atherosclerotic lesions. Representative images of oil-red O-stained aortas and quantification of plaque size. K, Quantification of blood Ly-6Chigh monocytes, Ly-6Clow monocytes and neutrophils. n=8 Ldlr−/− mice treated with exendin-4; n=6 Ldlr−/− mice treated with PBS; two-tailed unpaired Student's t-test. Data are mean±s.e.m. *P<0.05, **P<0.01, ***P<0.001.

FIGS. 13A-13F show gut intraepithelial Glp1r^{high} IELs regulate the bioavailability of GLP-1. A, Immunohistochemistry and quantification of GLP-1-producing L-cells in whole ileum preparations of 6 wild-type and 5 β7−/− mice. B, Small-intestinal IEL mixtures were incubated with the fluorescently (Cys40SeTau647) labelled GLP-1R agonist exendin-4 and the capacity of agonist binding by the different subsets—natural IELs (Glp1r^{high}), induced IELs (Glp1r^{low}) and non-T cells—was analysed by flow cytometry. Sorted Glp1rhigh and Glp1r^{low} cells were also incubated with recombinant GLP-1 and the remaining supernatant GLP-1 was plotted against the relative Glp1r mRNA levels of the cells. C, GLP-1—producing GLUTag cells were co-cultured with sorted natural (Glp1r^{high}) or induced (Glp1r^{low}) IELs. After 24 h, the concentration of GLP-1 in the supernatant was measured. n=5 biologically independent samples for Glp1r^{high} IELs and n=4 biologically independent samples for Glp1r^{low} IELs. D, Left, GLUTag cells were co-cultured with sorted Glp1r^{high} IELs in the presence of exendin-4 (100 nM) or control. n=3 independent biologically samples per group. Right, GLUTag cells were stimulated with exendin-4 (100 nM) or control. n=4 independent biological samples per group. After 24 h, the concentration of GLP-1 in the supernatant was measured. E, Sorted Glp1r^{high} IELs were incubated with exendin-4 (100 nM) or control. After 24 h, samples were centrifuged and supernatants were transferred to ex vivo ileum fractions of wild-type mice. GLP-1 levels were determined 24 h later from ex vivo supernatants. n=10 biologically independent mice per group. F, Whole gut preparations of wild-type or β7−/− mice were treated with or without the GLP-1 receptor antagonist exendin-9 (100 nM). After 24 h, the concentration of GLP-1 in the supernatant was measured. n=5 biologically independent samples for wild-type or β7−/− mice without exendin-9; n=4 biologically independent samples for wild-type mice with exendin-9. Data are mean±s.e.m. *P<0.05, **P<0.01. All P values from two-tailed unpaired Student's t-test.

FIG. 14 is a model. Without wishing to be bound by theory, it is proposed that integrin β7-dependent Glp1r^{high} IELs that reside in the small intestine modulate dietary metabolism by restricting the bioavailability of GLP-1.

### DETAILED DESCRIPTION

### Intraepithelial T Cells in the Gut

The biochemical response to food intake must be precisely regulated. Because ingested sugars and fats can feed into many anabolic and catabolic pathways (Begg DP & Woods SC The endocrinology of food intake. Nat Rev Endocrinol 9, 584-597 (2013)), how mammalian bodies handle nutrients depends on strategically positioned metabolic sensors that link the intrinsic nutritional value of a meal with intermediary metabolism.

The epithelium of the intestine digests and absorbs nutrients and fluids, and in adult humans it spans an area of about 200-400 m² (Cheroutre H, Lambolez F, Mucida D. Nat Rev Immunol. 2011 Jun. 17; 11(7):445-56). This huge surface is made up of a single cell layer of epithelial cells, which lines the lumen of the intestine to form a physical barrier between the core of the body and the environment and forms the largest entry port for pathogens. Integrin β7+ natural gut intraepithelial T lymphocytes (natural IELs) are dispersed throughout the enterocyte layer of the small intestine, and are known to form the front line of immune defense against invading pathogens (Cheroutre H, Lambolez F, Mucida D. Nat Rev Immunol. 2011 Jun. 17; 11(7):445-56). Despite their location in the gut, little is known about the role that integrin β7+ natural gut intraepithelial T lymphocytes play in metabolism. As such, the inventors of the present invention sought to determine the role that these lymphocytes play in metabolism and whether these cells could serve as a new target in the treatment of diseases related to metabolic syndrome.

### Metabolic Syndrome

Given the location of integrin β7+ natural gut intraepithelial T lymphocytes in the gut, the potential role of these natural IELs in metabolism was questioned. As shown herein, integrin β7− mice that lack natural IELs were metabolically hyperactive and, when fed a high-fat and high-sugar diet, were resistant to obesity, hypercholesterolaemia, hypertension, diabetes and atherosclerosis. Additionally shown herein, CCR9 knock out mice exhibited similar results to integrin β7− mice. Furthermore, shown here is that protection from cardiovascular disease in the absence of natural IELs depends on the enteroendocrine-derived incretin GLP-1 (Drucker, D. J. The cardiovascular biology of glucagon-like peptide-1. Cell Metab. 24, 15-30 (2016).), which is normally controlled by IELs through expression of the GLP-1 receptor. In this metabolic control system, IELs modulate enteroendocrine activity by acting as gatekeepers that limit the bioavailability of GLP-1. Although the function of IELs may prove advantageous when food is scarce, present-day overabundance of diets high in fat and sugar renders this metabolic checkpoint detrimental to health.

Accordingly, our identification that integrin β7+ natural gut intraepithelial T lymphocytes play a role in metabolic pathways highlights an important opportunity for targeting these T lymphocytes for the treatment of diseases resulting from metabolic syndrome.

Therefore, provided herein are methods of treating metabolic syndrome, e.g., by targeting integrin β7+ natural gut intraepithelial T lymphocytes, such as through the inhibition of integrin β7, inhibition of CCR9, or through the use of GLP-1 analogs, in a subject in need thereof. In some embodiments, the subject does not have multiple sclerosis, Crohn's disease, ulcerative colitis, or inflammatory bowel disease.

Metabolic syndrome has its general meaning in the art and includes numerous conditions that affect the heart, heart valves, blood, and vasculature of the body. Diseases associated with metabolic syndrome include endothelial dysfunction, coronary artery disease, angina pectoris, myocardial infarction, atherosclerosis, congestive heart failure, hypertension, cerebrovascular disease, stroke, transient ischemic attacks, deep vein thrombosis, peripheral artery disease, cardiomyopathy, arrhythmias, aortic stenosis, and aneurysm. Such diseases frequently involve atherosclerosis. Atherosclerosis is the root cause of myocardial infarction. Provided herein, in some embodiments, is blocking of integrin β7. Blocking of integrin β7 leads to resistance to obesity, hypertension, hypercholesterolemia, and indices of type II diabetes (specifically glucose tolerance).

Individuals with metabolic syndrome typically have or are at risk for high blood pressure, or hypertension; atherosclerosis or blockages in the arteries; high blood cholesterol, or hyperlipidemia; diabetes; obesity; chronic obstructive pulmonary disorder or other forms of reduced lung function; among others. In some embodiments, provided herein are methods for determining how to choose patients for treatment, such as through clinical trials. As a first step, we could focus on patients with obesity and type II diabetes and test whether blocking IEL leads to weight reduction or improvement of glucose tolerance. Testing improvement of atherosclerosis would be more difficult to do. However, we could also test whether blocking IELs reduces hypercholesterolemia.

Recognized risk factors for metabolic syndrome include age, sex, family history (including having a genetic predisposition for developing metabolic syndrome or diseases associated with metabolic syndrome), hypertension, dysglycemia, dyslipidemia, smoking, abdominal obesity (measured by waist circumference, for instance >102 cm in men and >88 cm in women), high BMI (e.g., BMI>30), insulin resistance, inflammation as measured by high-sensitivity C-reactive protein (hsCRP) levels, lack of consumption of fruits and vegetables, sedentary lifestyle, and psychosocial stress.

In some embodiments, the disease associated with the metabolic syndrome is a disease associated with atherosclerosis. Atherosclerosis, also referred to arteriosclerosis, is characterized by plaque (caused by fats, cholesterol, and other substances) buildup inside arteries, which eventually limits the flow of oxygen-rich blood to organs and other parts of the body. Atherosclerosis can lead to serious problems, including heart attack, stroke, or even death.

Typical medications for the treatment of heart diseases associated with metabolic syndrome include antiplatelet medications, which help prevent the build-up of plaque or help prevent blood clots; statins, which lower cholesterol; and angiotensin-converting enzyme (ACE) inhibitors, which help lower blood pressure. While these treatment options help in reducing the heart condition, patients usually have to combine these pharmacological treatments with lifestyle changes, and sometimes even surgical interventions. The present methods can be used in combination with any one or more of these treatments.

### Inhibitors of Integrin β7

The present methods can include the selection and/or administration of a treatment including an integrin β7 inhibitor. As noted, integrin β7 is necessary for the T cell homing. Specifically, integrin β7 is needed for IEL recruitment. Therefore, integrin β7 inhibitor has the effect of preventing recruitment of natural intraepithelial T lymphocyte (also referred to herein as β7+ natural intraepithelial T lymphocytes, intraepithelial T lymphocytes, or IELs) to the small intestine.

Specific exemplary β7 integrin antagonists include an antibody that specifically binds β7 integrin subunit, such as a humanized monoclonal antibody that specifically binds β7 integrin subunit. In several examples that antibody is a humanized form of HP2/1 monoclonal antibody, humanized form of L25 monoclonal antibody, humanized form of FIB504 monoclonal antibody, humanized form of FIB27 monoclonal antibody, humanized form of 2B4-3 monoclonal antibody or fragments thereof. In an example, the antagonist is Natalizumab (also known as TYSABRI™ or ANTEGRIN™). In another example, the antagonist is Vedolizumab (also known as ENTYVIO™). In another example, the antagonist is Etrolizumab. (RG7413). In another example, the antagonist is PN-943. In another example, the antagonist is Abrilumab (AMG 181)

### Inhibitors of CCR9

CC chemokine ligand 25 (CCL25), originally described as thymus-expressed chemokine (TECK), plays a crucial role in T cell homing to the small intestine via signaling through CC chemokine receptor 9 (CCR9). CCL25 is constitutively expressed within the small intestine, especially in epithelial crypts, while being weakly or not all in the colon and at other mucosal surfaces. CCR9 is the only known receptor for TECK/CCL25.

Desensitization of CCR9 or anti-TECK/CCL25 could attenuate the recruitment of lymphocytes to the microvessels of small intestine. Thus, the targeted blockade of CCL25-CCR9 interactions would inhibit the recruitment of integrin β7+ natural gut intraepithelial T lymphocytes, thereby providing an effective therapeutic treatment for metabolic syndrome.

The term “C-C chemokine receptor 9 inhibitor,” “CCR9 inhibitor” or “CCR9 chemokine receptor inhibitor” refers to an inhibitor or antagonist of a CCR9 receptor polypeptide, variants thereof, or fragments thereof.

Inhibition of the CCR9 chemokine receptor may be accomplished by the use of a small molecule compound. For instance, GSK-1605786 (CCX282; Traficet-EN), a selective antagonist of the CC chemokine receptor (CCR9), or CCX025. Further, PCT Published Application WO 2003/099773 (Millennium Pharmaceuticals, Inc.), U.S. Publication No. 2017/0216295, and U.S. Pat. No. 8,178,699 are exemplary disclosures describing compounds which can bind to and modulate CCR9 receptors.

### GLP-1 Agonists

Glucagon-like peptide-1 (GLP-1) is a 30 or 31 amino acid long peptide hormone deriving from the tissue-specific posttranslational processing of the proglucagon peptide. It is produced and secreted by intestinal enteroendocrine L-cells and certain neurons within the nucleus of the solitary tract in the brainstem upon food consumption. The initial product GLP-1 (1-37) is susceptible to amidation and proteolytic cleavage which gives rise to the two truncated and equipotent biologically active forms, GLP-1 (7-36) amide and GLP-1 (7-37). Active GLP-1 composes two a-helices from amino acid position 13-20 and 24-35 separated by a linker region. GLP-1 is an incretin; thus, it has the ability to decrease blood sugar levels in a glucose-dependent manner by enhancing the secretion of insulin. Beside the insulinotropic effects, GLP-1 has been associated with numerous regulatory and protective effects.

GLP-1 receptor agonists have been developed to increase GLP-1 activity. GLP-1-based treatment has been associated with weight loss and a lower risk of hypoglycemia, two important considerations for patients with type 2 diabetes.

GLP-1 agonists are well-known and have been described, for instance, to be useful for treating hyperglycemia (WO 98/08871), for treating dyslipidemia (WO 01/66135), for reducing morbidity and mortality after myocardial infarct (MI) (U.S. Pat. No. 6,277,819), for treating acute coronary syndrome (ACS), unstable angina (UA), non-Q-wave cardiac necrosis (NQCN) and Q-wave MI (QMI) (WO 01/89554), for reducing morbidity and mortality after stroke (WO 00/16797) as well as for increasing urine flow (WO 99/40788). PCT publications WO 98/08871 and WO 99/43706 disclose stable derivatives of GLP-1 analogues, which have a lipophilic substituent. These stable derivatives of GLP-1 analogues have a protracted profile of action compared to the corresponding GLP-1 analogues. Small non-peptidyl organic molecules are also known to be GLP-1 agonists.

GLP-1 agonists have well known effects on blood glucose and plasma lipids. GLP-1 agonists are potential drugs for the treatment and prevention of a wide range of cardiac and cardiovascular diseases.

As mentioned above, shown herein, IELs modulate enteroendocrine activity by acting as gatekeepers that limit the bioavailability of GLP-1. Accordingly, the present methods can include the use of a GLP-1 agonist or a pharmaceutically acceptable salt thereof for the preparation of a pharmaceutical composition for the treatment or reduction of risk of developing of an early cardiac or early cardiovascular disease in a patient in need thereof. By an early cardiac or early cardiovascular disease is meant a stage of disease prior to stroke or myocardial infarct.

Within the context of the present invention, “a GLP-1 agonist” is understood to refer to any compound, including peptides and non-peptide compounds, which fully or partially activates the human GLP-1 receptor.

Examples of GLP-1 agonists include, but are not limited to, Dulaglutide, Exenatide, Liraglutide, Lixisenatide, Insulin degludec/liraglutide, Insulin glargine/lixisenatide, Semaglutide.

Provided herein, in some embodiments, are methods for treating patients with a GLP-1 agonist who have hypertension but don't yet have diabetes or atherosclerosis. Or, in some embodiments, treating obese patients who don't yet have diabetes or atherosclerosis.

### General Definitions

As used herein, the term “patient” or “subject” refers to members of the animal kingdom including but not limited to human beings and “mammal” refers to all mammals, including, but not limited to human beings.

As used herein, “treating” or “preventing” metabolic syndrome or a disease associated with metabolic syndrome means administration to a patient by any suitable dosage regimen, procedure and/or administration route of a composition, device or structure with the object of achieving a desirable clinical/medical end-point, including but not limited to, stopping or slowing progression, reversing, or reducing the rate or risk of development of metabolic syndrome or a disease associated with metabolic syndrome. In some embodiments, the methods reduce localization of natural IELs to the small intestine.

### Pharmaceutical Compositions and Methods of use

Pharmaceutical compositions comprising a β7 integrin inhibitor, CCR inhibitor, or GLP-1 agonist may be prepared by any method known in the art of pharmacy, for example by bringing into association the active ingredient with the carrier(s) or excipient(s). As used herein, “carrier” or “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it may be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the β7 integrin inhibitor, CCR inhibitor, or GLP-1 agonist. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid.

Pharmaceutical compositions are typically formulated to be compatible with its intended route of administration. Examples of routes of administration include parenteral, e.g., intravenous, intradermal, subcutaneous, oral (e.g., inhalation), transdermal (topical), transmucosal, and rectal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, N.Y.). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

In some embodiments, provided herein are pharmaceutical compositions targeted for specific areas of the gastrointestinal tract, such as the stomach, small intestine, or colon. Delivery depends on withstanding the various pH's encountered in the GI tract. Delivery systems are described in, e.g., U.S. Pat. No. 6,531,152. For instance, delivery to the small intestine can include pharmaceutical compositions in which the drug is provided with an enteric coating. This coating protects the gastric mucosa from drug irritation. Coating is done with a selectively insoluble substance, and protects drugs from inactivation by gastric enzymes and/or low pH. Another method of drug targeting to the small intestine is drug absorption via the lymphatic system. Capillary and lymphatic vessels are permeable to lipid-soluble compounds and low molecular weight moieties (Magersohn, M., Modern Pharmaceutics, Marcel Dekker, New York (1979), pp. 23-85) (Ritschel, W. A., Meth Find Ex. Clin. Pharmacol 13(5):313-336 (1991)). Macromolecules, such as peptides, are absorbed into the lymphatics through Peyer's patches, which occur equally throughout all segments of the small intestine. Another approach for targeting drugs to the small intestine involves the use of intestinal sorption promoters. Studies have been carried out using long chain fatty acids, including linoleic acid, acylcarnitines, and palmitocamitine (Morimoto, K., et. al., Int. J. Pharmaceut. 14: 49-57 (1983); Fix, J. A., et. al., Aires J. Physiol. 14:G-332-40 (1986)). Alternatively, the pharmaceutical compositions provided herein may be administered intraperitoneally or subcutaneously. Many methods for the preparation of such formulations are known to those skilled in the art.

Additionally, β7 integrin inhibitor, CCR inhibitor, or GLP-1 agonist-containing compositions may be in variety of forms. The preferred form depends on the intended mode of administration and therapeutic application, which will in turn dictate the types of carriers/excipients. Suitable forms include, but are not limited to, liquid, semi-solid and solid dosage forms.

A “therapeutically effective amount” refers to an amount effective, at dosages and for periods of time necessary, to achieve the desired therapeutic result. An “amount effective” for treatment of a condition is an amount of an active agent or dosage form, such as the coacervate composition described herein, effective to achieve a determinable end-point. The “amount effective” is preferably safe—at least to the extent the benefits of treatment outweighs the detriments and/or the detriments are acceptable to one of ordinary skill and/or to an appropriate regulatory agency, such as the U.S. Food and Drug Administration.

Compositions comprising the agents of the present invention can be formulated into a composition in a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The compositions may comprise an antibody. The term “antibody” as used herein refers to an immunoglobulin molecule or an antigen-binding portion thereof. Examples of antigen-binding portions of immunoglobulin molecules include F(ab) and F(ab′)2fragments, which retain the ability to bind antigen. The antibody can be polyclonal, monoclonal, recombinant, chimeric, de-immunized or humanized, fully human, non-human, (e.g., murine), or single chain antibody. In some embodiments the antibody has effector function and can fix complement. In some embodiments, the antibody has reduced or no ability to bind an Fc receptor. For example, the antibody can be an isotype or subtype, fragment or other mutant, which does not support binding to an Fc receptor, e.g., it has a mutagenized or deleted Fc receptor binding region. Methods for making antibodies and fragments thereof are known in the art, see, e.g., Harlow et. al., editors, Antibodies: A Laboratory Manual (1988); Goding, Monoclonal Antibodies: Principles and Practice, (N.Y. Academic Press 1983); Howard and Kaser, Making and Using Antibodies: A Practical Handbook (CRC Press; 1st edition, Dec. 13, 2006); Kontermann and Dübel, Antibody Engineering Volume 1 (Springer Protocols) (Springer; 2nd ed., May 21, 2010); Lo, Antibody Engineering: Methods and Protocols (Methods in Molecular Biology) (Humana Press; Nov. 10, 2010); and Dübel, Handbook of Therapeutic Antibodies: Technologies, Emerging Developments and Approved Therapeutics, (Wiley-VCH; 1 edition Sep. 7, 2010).

Dosage regimens may be adjusted to provide the optimum desired response (e.g., a therapeutic or prophylactic response). For example, a single bolus may be administered, several divided doses may be administered over time, or the composition may be administered continuously or in a pulsed fashion with doses or partial doses being administered at regular intervals, for example, ever 10, 15, 20, 30, 45, 60, 90, or 120 minutes, every 2 through 12 hours daily, or every other day, etc. be proportionally reduced or increased as indicated by the exigencies of the therapeutic situation. In some instances, it may be especially advantageous to formulate parenteral compositions in dosage unit form for ease of administration and uniformity of dosage. The specification for the dosage unit forms of the invention are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

### EXAMPLES

The invention is further described in the following examples, which do not limit the scope of the invention described in the claims.

### Materials and Methods

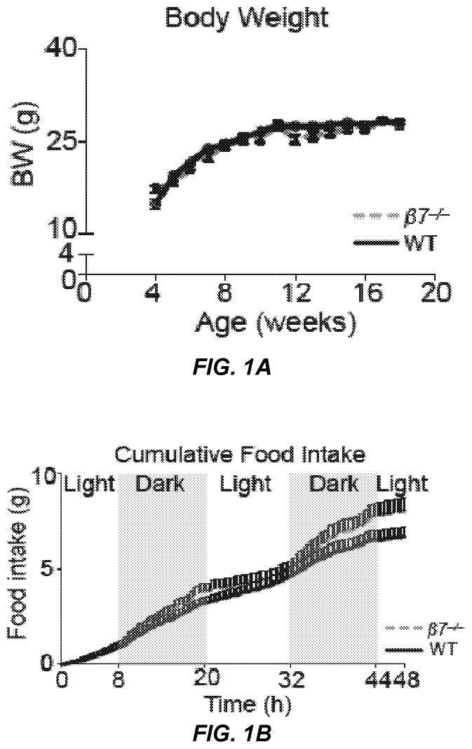

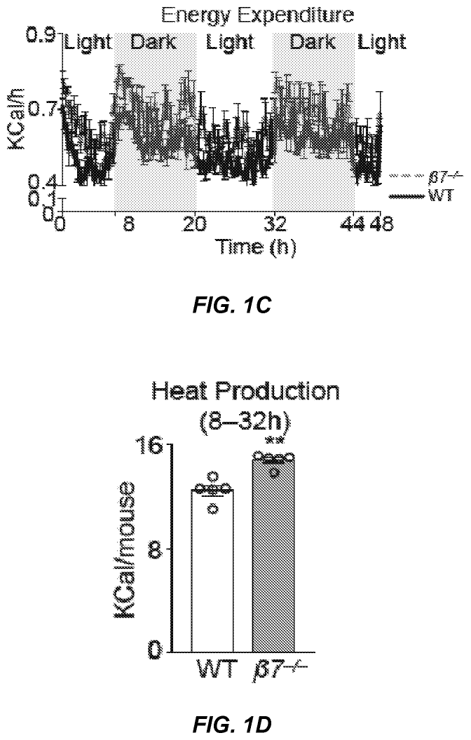

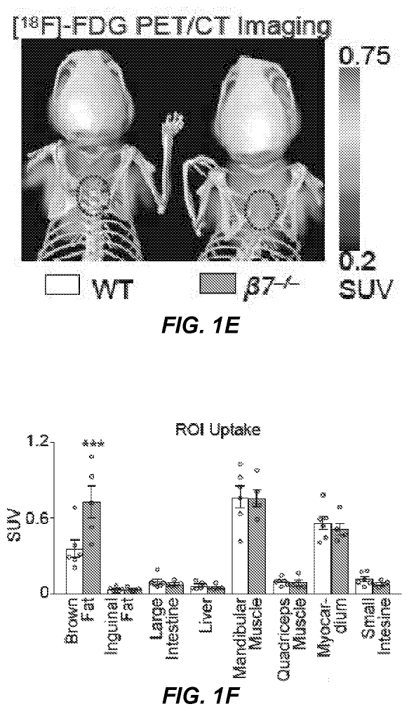

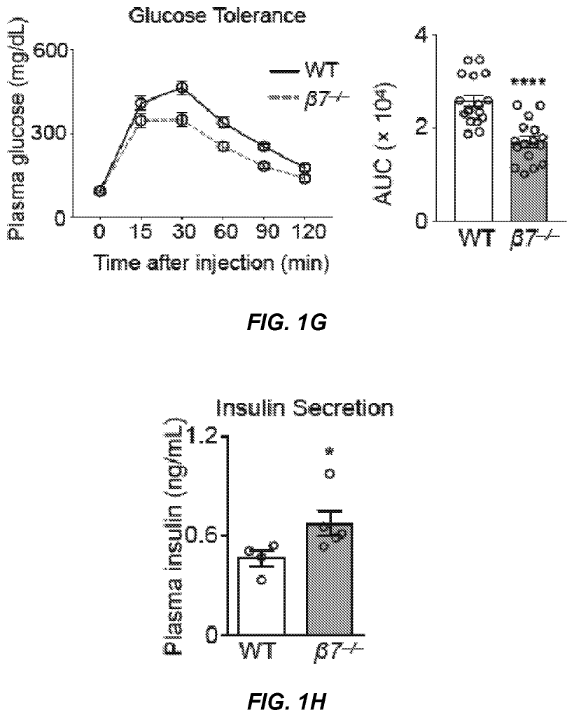

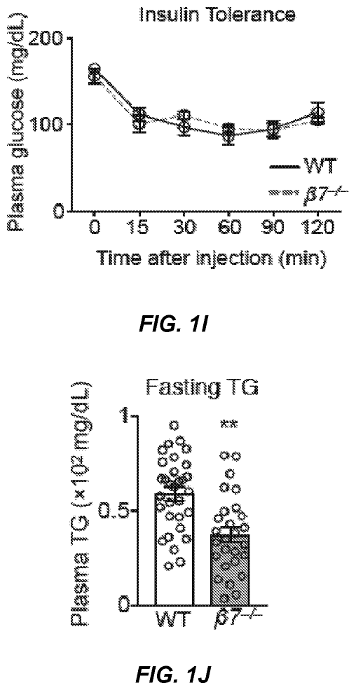

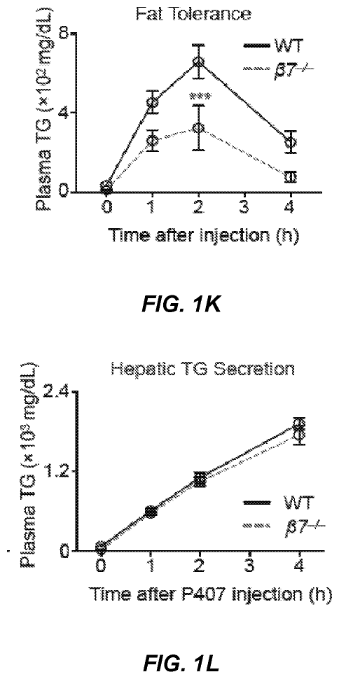

Mice. C57BL/6J (wild-type mice), B6.SJL-PtprcaPepcb/BoyJ (CD45.1+), Itgb7tm1Cgn (β7−/−), Ldlrtm1Her (Ldlr−/−), Tcrbtm1Mom (βTCR−/−), Tcrdtm1Mom (γδTCR/), Ccr2tm1Ifc (Ccr2−/−), Ighmtm1Cgn (μMT), Ccr9tm1Lov (Ccr9−/−), Itgaetm1Cmp (Itgae−/−), and C57BL/6-Tg(UBC-GFP)30Scha/J (GFP+) mice were purchased from The Jackson Laboratory. Glp1r−/− mice on the C57BL/6 background were bred in-house as described30. Unless otherwise indicated, age- and sex-matched animals were used starting at 8-12 weeks of age. Female mice were used for experiments in FIGS. 1H, 1K, 1L, 4E, 5G, 8C, 8D. A mixture of sexes was used in FIG. 1J. Male mice were used in all other experiments. Investigators were blinded to group allocation during data collection and analysis. Where appropriate, animals were randomly assigned to interventions. All protocols were approved by the Animal Review Committee at Massachusetts General Hospital (protocols 2011N000035 and 2015N000044) and were in compliance with relevant ethical regulations.

### Animal Models and in Vivo Interventions

Diet. For studies on metabolic syndrome, wild-type and β7−/− mice were fed a HFSSD (Research Diets D12331). For studies on atherosclerosis, bone marrow chimaeras on the Ldlr−/− background or double knockouts were fed a HCD (Research Diets D12108C). HFSSD-fed and HCD-fed mice were single- or group-housed on a 12-h:12-h light:dark cycle at 22° C. with free access to food and water. HFSSD-fed mice were maintained under these conditions for 21 weeks and HCD-fed mice for 14 weeks. For studies on glucose tolerance in thermoneutrality, wild-type mice and β7−/− mice were housed for 3 days in a 12-h:12-h light:dark cycle at 30° C. with free access to food and water. For studies on microbiota, mice were treated with an antibiotics cocktail (0.1% ampicillin, 0.1% metronidazole, 0.05% vancomycin and 0.1% neomycin) in drinking water for 4 weeks and an equal number of 8-week-old wild-type and 7−/− mice with the same day of birth were co-housed for 4 weeks.

Bone marrow transplantation. Naive C57BL/6 or Ldlr−/− mice were lethally irradiated (950 cGy) and reconstituted with indicated bone marrow cells to generate different chimaera groups: (i) bmβ7+/+Ldlr−/− and bmβ7−/−Ldlr−/− (irradiated Ldlr−/− mice reconstituted with either wild-type or β7−/− bone marrow); (ii) bmβ7+/+ CD45.1+ or bmβ7−/−GFP+Ldlr−/−(irradiated Ldlr−/− mice reconstituted with a 1:1 bone marrow mixture of CD45.1+ wild-type and GFP+β7−/− bone marrow); (iii) bmβ7+/+CD45.1+ or bmβ7−/−GFP+ (irradiated wild-type mice reconstituted with a 1:1 bone marrow mixture of CD45.1+ wild-type and GFP+β7−/− bone marrow); (iv) β7−/− wild-type, β7−/−βTCR−/−, and β7−/−γδTCR−/− (irradiated wild-type mice reconstituted with 1:1 bone marrow mixtures of β7−/− and wild-type, β7−/− and βTCR−/−, and β7−/− and γδTCR−/−, respectively); (v) bmGlp1r+/+β7−/−Ldlr−/− and bmGlp1r−/−β7−/−Ldlr−/− (irradiated Ldlr−/− mice reconstituted with 1:1 bone marrow mixtures of wild-type and β7−/−, and Glp1r−/− and β7−/−, respectively); (vi) bmGlp1r+/+ and bmGlp1r−/− (irradiated wild-type mice reconstituted with either wild-type or Glp1r−/− bone marrow); and (vii) bmGlp1r+/+GFP+ and bmGlp1r−/−GFP+ (irradiated wild-type mice reconstituted with 1:1 bone marrow mixtures of wild-type and GFP+, and Glp1r−/− and GFP+, respectively); (viii) bmβ7−/−Ccr2−/−, bmβ7−/− μMT, and bmβ7−/− wild-type (irradiated wild-type mice reconstituted with 1:1 bone marrow mixtures of β7−/− and Ccr2−/−, β7−/− and μMT, and β7−/− and wild-type, respectively; (ix) bmβ7−/− wild-type Ldlr−/− or bmβ7−/− μMT Ldlr−/− (irradiated Ldlr−/− mice reconstituted with 1:1 bone marrow mixtures of β7−/− and wildtype, and β7−/− and μMT, respectively.

Anti-integrin β7 antibody treatment. Ldlr−/− mice on a HCD were treated with anti-integrin β7 antibodies (FIB504, BioxCell) or IgG isotype control (2A3, BioxCell) by intraperitoneal injection for 14 weeks for quantification of atherosclerotic plaque size after euthanasia (500 μg per mouse per week).

Glp1r agonist treatment. Ldlr−/− mice were treated with the Glp1r agonist exendin-4 (Abcam) at a dose of 100 μg per kg per day via osmotic minipumps (Alzet). After 8 weeks on a HCD, mice were euthanized for atherosclerotic lesion quantification.

### Metabolic Measurements

CLAMS. A comprehensive laboratory animal monitoring system (CLAMS, The Columbus Instruments) was used at the Joslin Diabetes Center Animal Physiology Core to simultaneously measure a series of metabolic parameters including energy expenditure (heat production), oxygen consumption (VO2), carbon dioxide production (VCO2), respiratory exchange ratio, food consumption and locomotor activity levels.

Blood pressure measurements. Systolic and diastolic blood pressures were measured using a non-invasive tail-cuff system (Kent Scientific). Mice were initially acclimatized to the instrument for three consecutive days before the measurements. [¹⁸F]FDG PET/CT. The uptake and distribution of glucose in vivo were determined by [¹⁸F]FDG and non-invasive, high-resolution PET/CT imaging and ex vivo biodistribution. In brief, mice were anaesthetized with isoflurane and injected intravenously through the tail vein with around 200 μCi of tracer diluted to a final volume of 150 μl in isotonic saline. Following a 60-min absorption period, mice were imaged on a Siemens Inveon small-animal integrated PET/CT scanner. The CT was acquired over 360 projections using a 80 kV, 1 μA X-ray tube operating at 80 kilovoltage peak (kVp) and 500 μA on a CMOS detector and reconstructed using a modified Feldkamp cone beam reconstruction algorithm (COBRA, Exxim Computing Corporation). A 20-min PET image was acquired and reconstructed using the ordered subsets expectation maximization followed by maximum a posteriori. Regions of interest were manually drawn for standard uptake value calculations. After imaging, animals were euthanized and tissues were collected for biodistribution analysis using gamma well counting on a 20% window on the 511 keV photopeak (Wizard2, PerkinElmer). [¹⁸F]FDG levels were normalized to the weight of resected tissue and expressed as the percentage injected dose per gram tissue weight.

Glucose- and insulin-tolerance tests. For glucose- and insulin-tolerance tests, overnight-fasted mice were injected intraperitoneally or by oral gavage with glucose (2 g per kg body weight) or injected intraperitoneally with insulin (0.75 U per kg body weight). Blood glucose levels were measured at the basal level and at 15, 30, 60, 90 and 120 min after glucose or insulin administration using One Touch Ultra2 Blood Glucose Meter (OneTouch, LifeScan).

Fat-tolerance test. Overnight-fasted mice were injected intraperitoneally with 200 μl 20% Intralipid (vol/vol) fat emulsion (Sigma, MA), and blood that was drawn from the retroorbital plexus at the indicated time points for triglyceride measurement using the L-Type Triglyceride M kit (Wako Diagnostics, VA). To measure hepatic lipid export, overnight-fasted mice were injected with 1 g per kg poloxamer 407 (Pluronic F-127, Sigma) and plasma was collected at the indicated time points for triglyceride analysis.

Fat absorption test. To measure fat absorption in the gut, overnight-fasted mice were injected intraperitoneally with 1 g per kg poloxamer 407. After 1 min, the mice were gavaged with 0.4 ml corn oil. Plasma was collected at baseline as well as at the indicated time points after gavage for analysis of triglyceride levels.

FPLC. Mice were fasted for 12-16 h overnight before blood samples were collected by retro-orbital venous plexus puncture, after which plasma was separated by centrifugation. The lipid distribution in plasma lipoprotein fractions was assessed by fast-performed liquid chromatography (FPLC) gel filtration with 2 Superose 6 HR 10/30 columns (Pharmacia Biotech). Total plasma cholesterol in each fraction was enzymatically measured (Wako Pure Chemicals).

Cholesterol. Plasma was collected from overnight-fasted or non-fasted mice that were fed a HCD for 14 weeks, and plasma total cholesterol was determined by a Cholesterol E kit (Wako Diagnostics).

Gut permeability test. Overnight-fasted mice were administered through oral gavage with fluorescein isothiocyanate (FITC)-dextran (Sigma-Aldrich) at a dose of 12 mg per mouse, and plasma was collected 4 h later for fluorescence intensity measurement. A mouse model of colitis in which a mouse was treated with dextran sulfate sodium salt (36-50 kDa, MP Biomedicals) in drinking water for 6 days was used as a positive control for gut barrier disruption.

### Cells

Cell collection. Peripheral blood was collected by retro-orbital bleeding and red blood cells were lysed in RBC lysis buffer (Biolegend). Aortas were excised after PBS perfusion (Thermo Fisher Scientific), minced and digested with 450 U ml-1 collagenase I, 125 U ml-1 collagenase XI, 60 U ml-1 DNase I and 60 U ml-1 hyaluronidase (Sigma-Aldrich) in PBS for 40 min at 37° C. Total viable cell numbers were counted using trypan blue (Cellgro, Mediatech). Small-intestine IELs were isolated as follows: after excision of the small intestine, the Peyer's patches were removed under a microscope and the gut was cut open longitudinally to wash off the lumen contents in HBSS buffer. The gut was then cut into 1-2-cm pieces and subjected to 3 Å˜dissociation in EDTA-containing buffer (7.5 mM HEPES, 2% FCS, 2 mM EDTA, 10,000 U ml-1 penicillin-streptomycin, 50 μg ml-1 gentamycin in HBSS; all Thermo Fisher Scientific) in a shaker at 37° C. for 15 min. After dissociation the IELs were collected by filtering the lamina propria through a mesh.

Cell sorting. The IEL flow through after dissociation was further subjected to Percoll (GE Healthcare Bio-Sciences) grade centrifugation to remove the mucus. Single-cell suspensions of IELs from indicated animals were then stained to identify indicated cell populations. Cells were sorted on a FACS Aria II cell sorter (BD Biosciences) directly into either RLT buffer for subsequent RNA isolation or into collection medium for ex vivo manipulations.

Flow cytometry. Single-cell suspensions were stained in PBS supplemented with sterile 2% FBS and 0.5% BSA. The following monoclonal antibodies were used for flow cytometric analysis: anti-integrin β7 (clone FIB27), anti-CD45 (30-F11), anti-CD45.1 (clone A20), anti-CD45.2 (clone 104), anti-CD3 (clone 17A2), anti-CD90.2 (clone 53-2.1), anti-CD19 (clone 6D5), anti-B220 (clone RA3-6B2), anti-NK1.1 (clone PK136), anti-Ly-6G (clone 1A8), anti-Ly-6C (AL-21), anti-MHCII (clone AF6-120.1), anti-F4/80 (clone BM8), anti-CD11b (clone M1/70), anti-CD5 (clone 53-7.3), anti-βTCR (clone H57-597), anti-γδTCR (clone GL3), anti-CD326 (clone G8.8), anti-IgA (C10-3), anti-IgD (11-26c.2a), anti-CD115 (clone AFS98), and anti-CX3CR1 (clone SA011F11). Antibodies were all purchased from BioLegend except anti-IgA (BD Biosciences). Viable cells were identified as unstained with Zombie Aqua (Biolegend). Cells were defined as: (i) Ly-6Chigh monocytes (CD45+Lin−CD11b+F4/80−Ly-6Chigh); (ii) neutrophils (CD45+CD11b+Lin+F4/80−); (iii) macrophages (CD45+Lin−CD11b+F4/80+Ly-6Clow); (iv) epithelial cells (CD45−CD326+CD3−); or (v) myeloid cells (CD45+CD3−CD11b+). Lineages were defined as: Lin: CD3, CD90.2, CD19, B220, NK1.1, Ter119, Ly-6G. Data were acquired on a LSRII (BD Biosciences) and analysed with FlowJo (Tree Star).

Cell culture. For all experiments, cells or ex vivo ileum tissues were kept in a humidified 5% CO2 incubator at 37° C. (i) For in vitro GLP-1 receptor agonist-binding experiments, small-intestinal IELs were isolated and incubated with 50 pM fluorescently labelled GLP-R agonist exendin-4 Cys40SeTau647 for 1 h, and IEL subsets were gated as follows: natural, Glp1r^{high} IELs (CD45+CD3+CD90.2−CD5−); induced, Glp1r^{low} IELs (CD45+CD3+CD90.2+CD5+), non-T cells (CD45+CD3−), and epithelial cells (CD45−CD326+CD3−). The binding capacity was analysed for exendin-4 Cys40SeTau647 by flow cytometry. (ii) For in vitro co-culture experiments, GLP-1-producing L-cells (GLUTag cells that were provided by D.J.D., authenticated multiple times and tested for mycoplasma) were cultured together with sorted Glp1r^{high} or Glp1r^{low} IELs in DMEM and GlutaMAX-I with glucose 1 gl-1 (Invitrogen) supplemented with 10% FBS and 1% penicillin-streptomycin (103 GLUTag cells and 105 IELs in a 96-well plate in 200 μl medium per well). After 24 h, the concentration of GLP-1 in the supernatant was measured using a total GLP-1 enzyme-linked immunosorbent assay (ELISA) kit (Millipore). In some experiments, the GLP-1 receptor agonist exendin-4 (Abcam) was added to co-culture wells (100 nM). (iii) In the case of two-step ex vivo experiments, sorted Glp1r^{high} IELs were incubated with exendin-4 (100 nM) or control. After 24 h, samples were centrifuged (300 g, 5 min) and supernatants were transferred to ileum ex vivo sections of previously euthanized wild-type mice. GLP-1 levels were determined 24 h later from ex vivo supernatants. (iv) In an ex vivo GLP-1 receptor-inhibition experiment, whole ileum preparations of wild-type or β7−/− mice were treated with the GLP-1 receptor antagonist exendin-9 (100 nM) or control. After 24 h the concentration of GLP-1 in the supernatant was measured using a total GLP-1 ELISA kit (Millipore).

### Molecular Biology

PCR Total RNA was isolated using the RNeasy Mini Kit (Qiagen) according to the manufacturer's instructions. cDNA was generated from 1 μg of total RNA per sample using the High Capacity cDNA Reverse Transcription Kit (Applied Biosystems). Quantitative real-time TaqMan PCR was performed using the following TaqMan primers (Applied Biosystems): Glp1r (Mm00445292_m1), Gcg (Mm00801714_m1) and housekeeping gene actin (Actb) (Mm02619580_g1). PCR was run on a 7500 thermal cycler (Applied Biosystems) and data were quantified with the 2-ΔCt method.

ELISA. Total GLP-1 levels were measured in plasma of overnight-fasted mice or during oral glucose-tolerance tests using a commercial ELISA kit (Millipore) according to the manufacturer's instructions. Gut IgA was retrieved by flushing the lumens of dissected guts with 5 ml PBS, and both gut flush IgA and plasma IgA was detected using mouse IgA ELISA kit (Bethyl Laboratories).

### Histology

Adipose tissue. iWAT and pWAT were excised, fixed in 10% formalin solution and paraffin-embedded. Haematoxylin and eosin staining was performed to assess overall tissue morphology. The adipocyte size distribution was determined with the NIH ImageJ program. Aortas. Aortic roots were dissected, embedded in Tissue-Tek OCT compound (Sakura Finetek) and frozen in 2-methylbutane (Fisher Scientific) cooled with dry ice. For comparisons of lesion sizes between the groups, sections with the maximum lesion area were used. To measure lesion volume, sections were collected at the first appearance of the aortic valves until lesions were no longer visible. Oil red O staining (Sigma-Aldrich) was performed to visualize lipid content and the lesion size was measured. To quantify lesion macrophage and smooth muscle cell content, immunohistochemistry was performed with anti-CD68 (BioLegend) and anti-Myh11 (Millipore) antibodies. The positive cells were visualized using the Vectastain ABC kit (Vector Laboratories) and AEC substrate (DAKO/Agilent Technologies) and the slides were counterstained with Harris haematoxylin (Sigma-Aldrich). To quantify collagen content, Masson trichrome staining (Sigma-Aldrich) was performed.

Ileum CD3 cell numbers. Small intestines were dissected and cut open. After rinsing away the lumen contents in PBS, ileum was rolled from proximal to distal parts and embedded for histological sectioning. Immunohistochemistry was performed using an anti-CD3 antibody (BioLegend) and CD3+ cells were quantified.

Ileum L-cell numbers. ileum sections of wild-type and β7−/− mice were paraffin-embedded and GLP-1 staining (Abcam) was performed to quantify GLP-1-producing L-cells in the entire ileum of each mouse. A biotinylated secondary antibody and streptavidin DyLight 594 (Vector Laboratories) were applied and nuclei were identified using DAPI (Thermo Fisher Scientific). All histological slides were scanned using a digital slide scanner NanoZoomer 2.0RS (Hamamatsu).

Statistics. Results are shown as mean±s.e.m. Unless indicated, statistical tests included unpaired, two-tailed Student's t-tests and nonparametric Mann-Whitney U-tests (when a Gaussian distribution was not assumed). For multiple-comparisons tests, nonparametric tests that compare the mean rank of each group (when a Gaussian distribution was not assumed) were performed. P values of 0.05 or less were considered to denote significance.

### Example 1

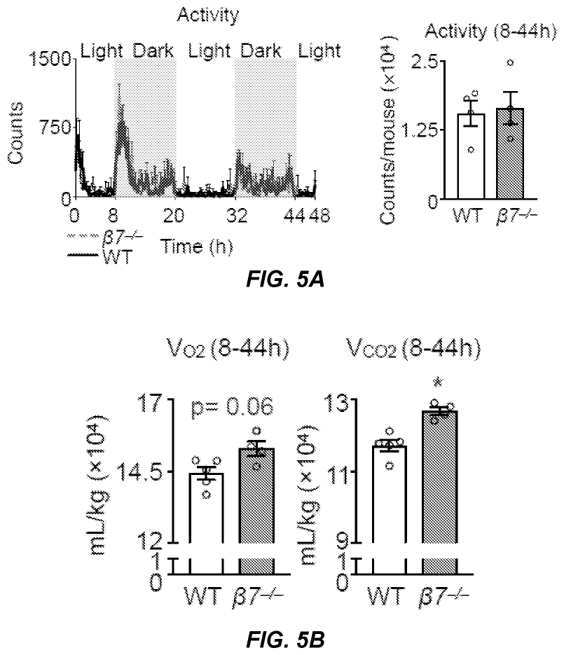

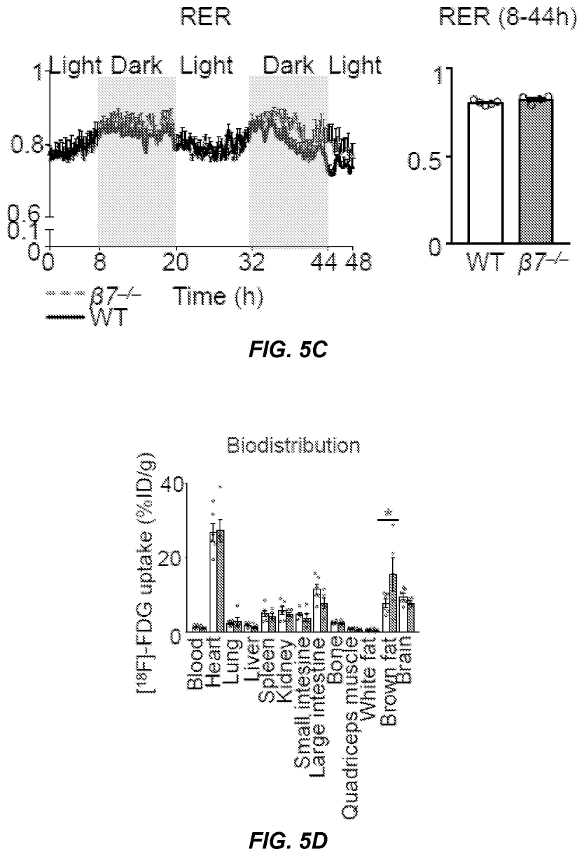

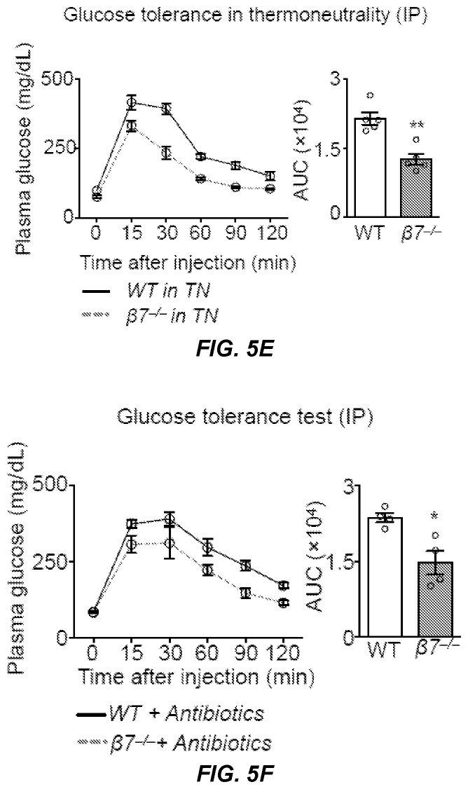

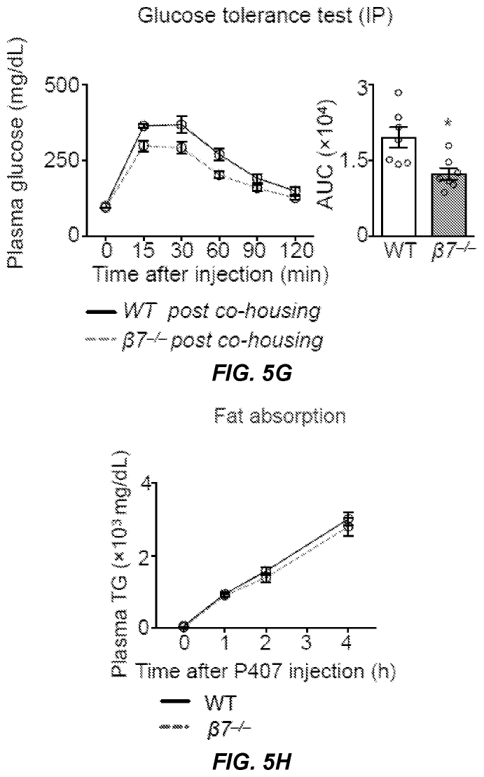

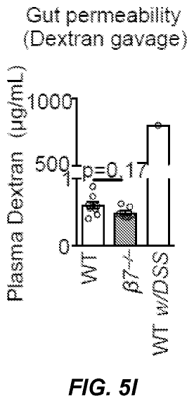

Although integrin β7 directs immune cells to the gut (Cerf-Bensussan, N., Begue, B., Gagnon, J. & Meo, T. The human intraepithelial lymphocyte marker HML-1 is an integrin consisting of a β7 subunit associated with a distinctive a chain. Eur. J. Immunol. 22, 273-277 (1992); Cheroutre, H., Lambolez, F. & Mucida, D. The light and dark sides of intestinal intraepithelial lymphocytes. Nat. Rev. Immunol. 11, 445-456 (2011); Holzmann, B., McIntyre, B. W. & Weissman, I. L. Identification of a murine Peyer's patch-specific lymphocyte homing receptor as an integrin molecule with an a chain homologous to human VLA-4α. Cell 56, 37-46 (1989); Holzmann, B. & Weissman, I. L. Peyer's patch-specific lymphocyte homing receptors consist of a VLA-4-like α chain associated with either of two integrin β chains, one of which is novel. EMBO J. 8, 1735-1741 (1989); Parker, C. M. et al. A family of β7 integrins on human mucosal lymphocytes.Proc. Natl Acad. Sci. USA 89, 1924-1928 (1992); Gorfu, G., Rivera-Nieves, J. & Ley, K. Role of β7 integrins in intestinal lymphocyte homing and retention. Curr. Mol. Med. 9, 836-850 (2009)), we know little about the influence of this integrin on metabolism, despite the strategic location of the gut as the site where dietary nutrients are absorbed (Buck, M. D., Sowell, R. T., Kaech, S. M. & Pearce, E. L. Metabolic instruction of immunity. Cell 169, 570-586 (2017)). Itgb7−/− (hereafter β7−/−) mice that were fed a chow diet gained weight in a similar way to control wild-type mice (FIG. 1A), but ate more food (FIG. 1B) despite being equally active (FIG. 5A). This inconsistency prompted us to measure energy usage. We found that β7−/− mice expended more energy (FIG. 1C) and produced more heat (FIG. 1D), although their respiratory exchange rate was similar to wild-type mice (FIGS. 5B, 5C). The data suggest that these mice have a heightened basal metabolism. We therefore performed whole-body 18F-fluorodeoxyglucose ([¹⁸F] FDG) and non-invasive, high-resolution positron emission tomography/computed tomography (PET/CT) imaging to assess regional glucose uptake and found that β7−/− mice accrued more glucose in the brown fat compared to wild-type controls (FIGS. 1E, 1F and FIG. 5D). The β7−/− mice were more glucose tolerant (FIG. 1G), even at thermoneutrality (FIG. 5E), and had higher levels of plasma insulin (FIG. 1H) without changes in insulin sensitivity (FIG. 1I). The microbiome did not appear to affect these differences (FIGS. 5F, 5G). Moreover, the phenomenon was neither restricted to glucose—because β7−/− mice had lower levels of fasting triglycerides (FIG. 1J) and better fat tolerance (FIG. 1K) without differences in hepatic secretion of triglycerides (FIG. 1L)—nor associated with fat absorption or gut permeability abnormalities (FIGS. 1H, 1I).

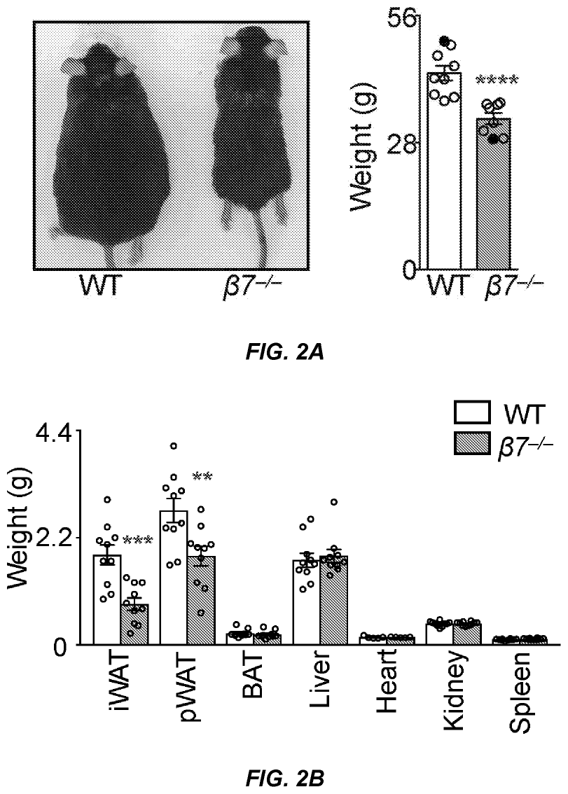

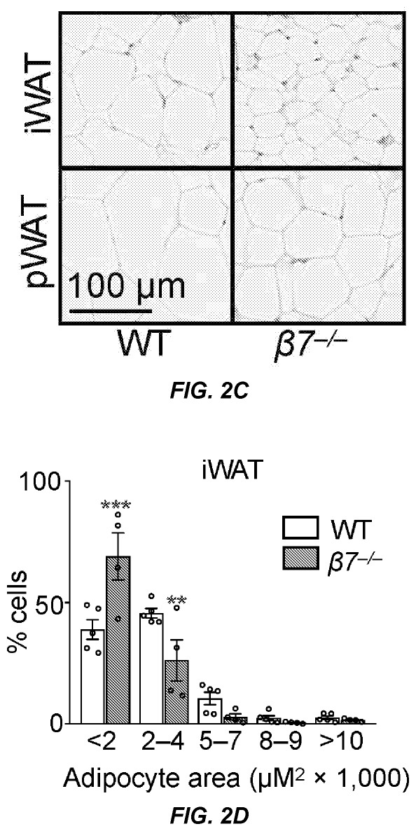

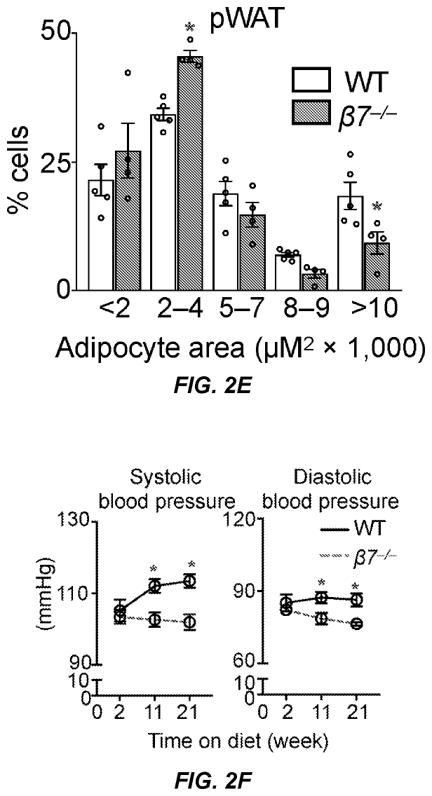

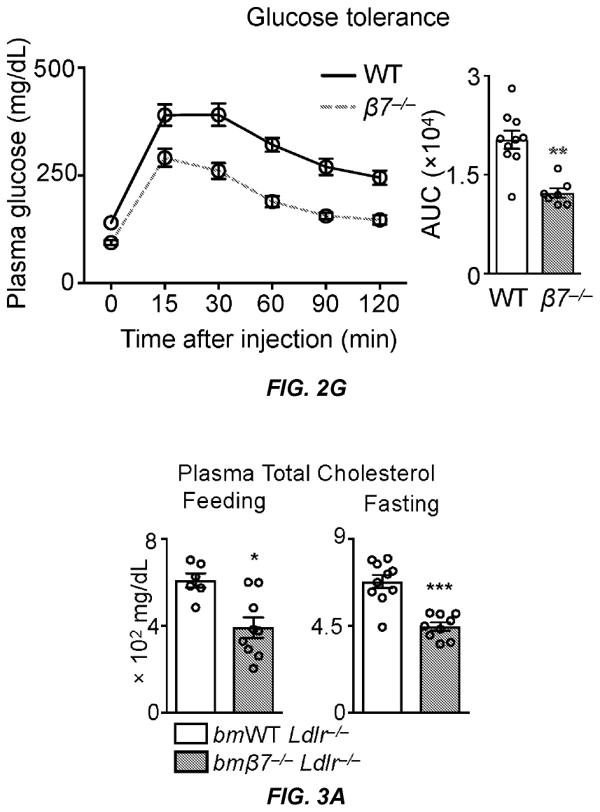

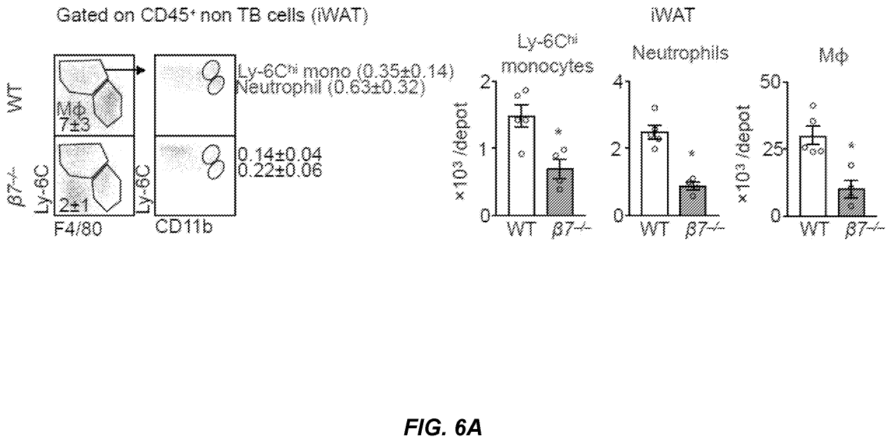

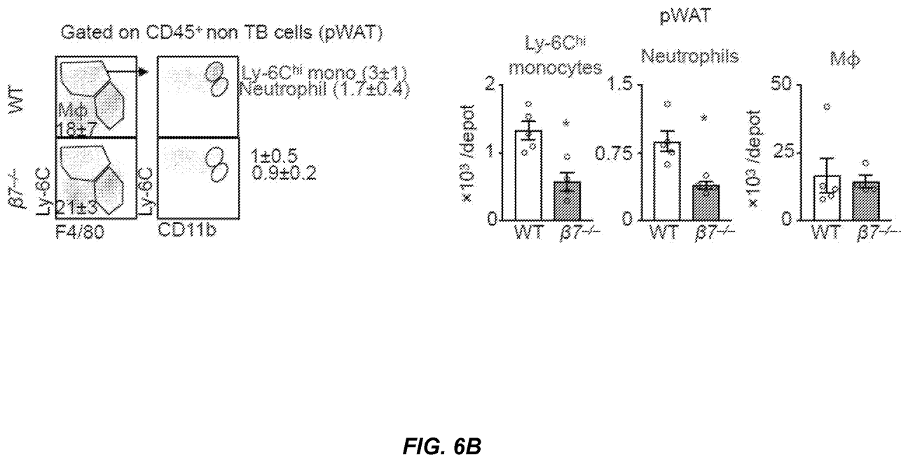

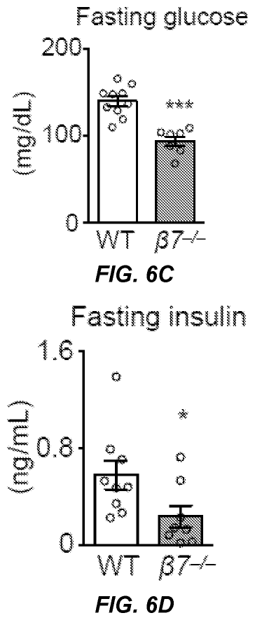

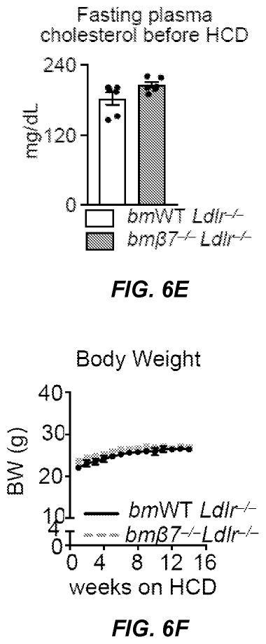

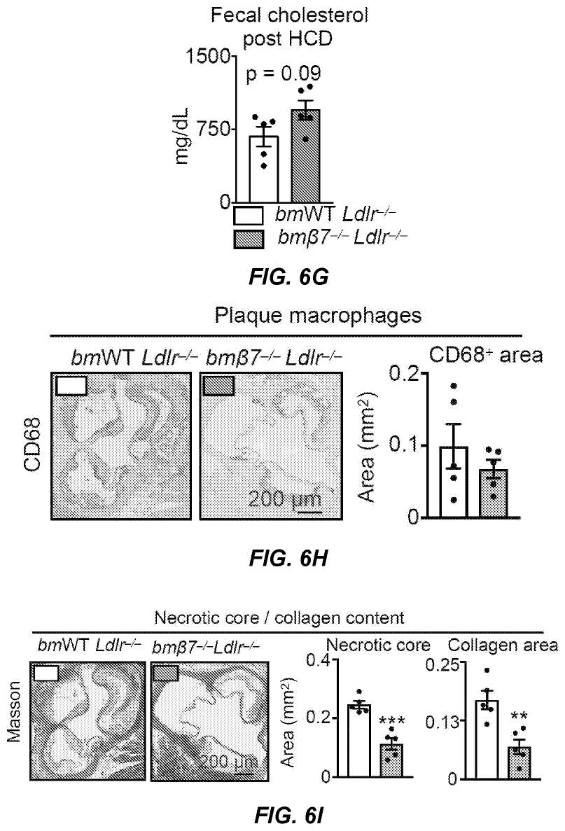

We next tested whether the beneficial metabolic alterations in β7−/− mice were sustained in the context of the ‘metabolic syndrome’ component cluster10. β7−/− mice that were fed a diet high in fat, sugar and sodium (HFSSD) remained relatively lean, in contrast to wild-type controls, which became obese (FIG. 2A). Both inguinal white adipose tissue (iWAT) and perigonadal white adipose tissue (pWAT) were heavier in wild-type mice than in β7−/− mice, but other tissue weights remained similar (FIG. 2B). Furthermore, adipocytes in iWAT and pWAT were larger in wild-type mice than in β7−/− mice (FIG. 2C-2E). Flow cytometry of both iWAT and pWAT showed that fewer Ly-6Chigh monocytes, neutrophils and macrophages had accumulated in β7−/− mice, indicating that β7−/− mice were protected from obesity-associated inflammation (Lumeng, C. N. & Saltiel, A. R. Inflammatory links between obesity and metabolic disease. J. Clin. Invest. 121, 2111-2117 (2011); Odegaard, J. I. & Chawla, A. The immune system as a sensor of the metabolic state. Immunity 38, 644-654 (2013)) (FIGS. 6A, 6B). In contrast to wild-type control mice, β7−/− mice did not develop hypertension (FIG. 2F) and—similar to observations made in β7−/− mice that were on a chow diet—β7−/− mice that were fed a HFSSD remained more glucose-tolerant than wild-type mice (FIG. 2G and FIGS. 6C, 6D), indicating that β7−/− mice were protected against the adverse metabolic consequences of a high-fat diet.

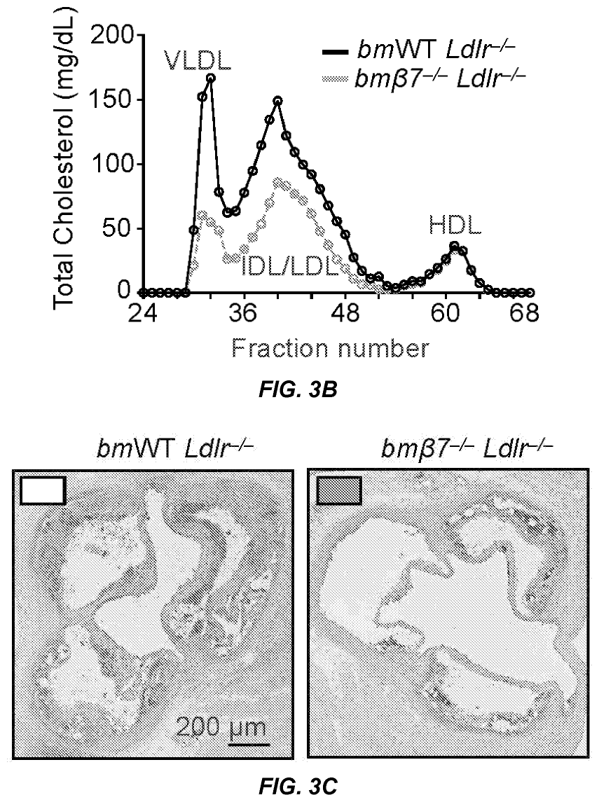

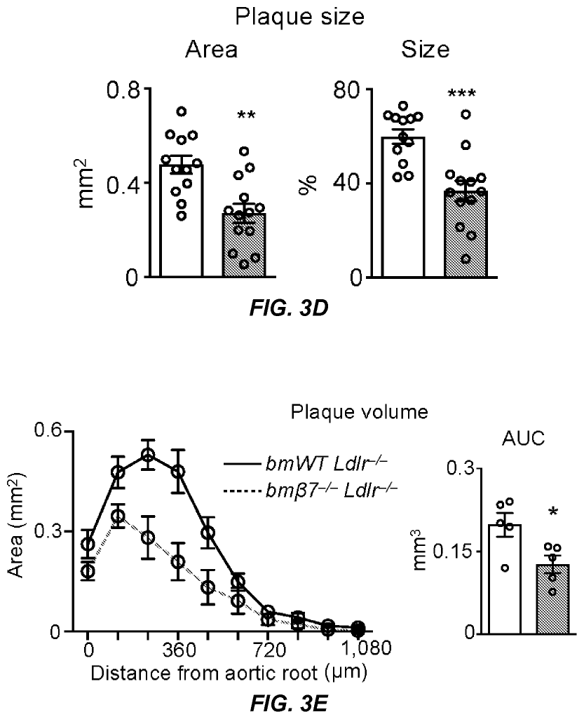

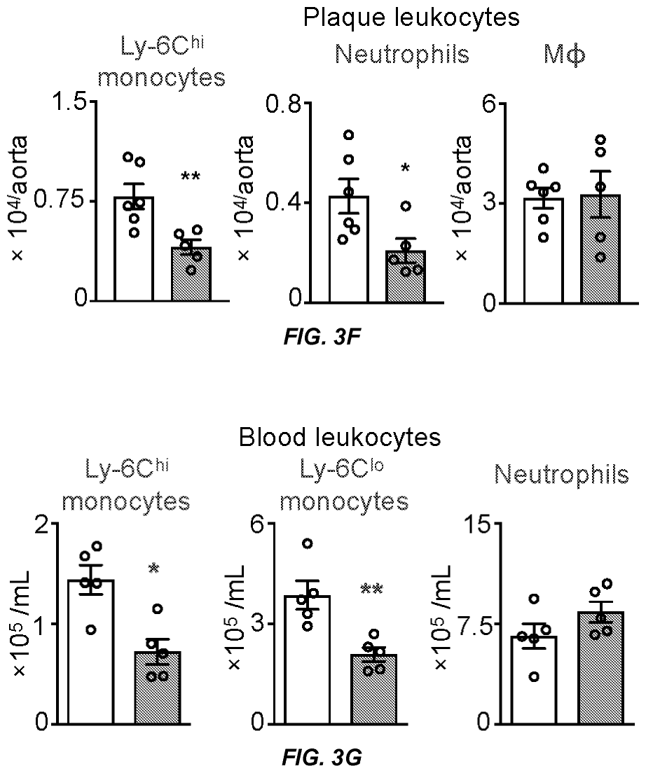

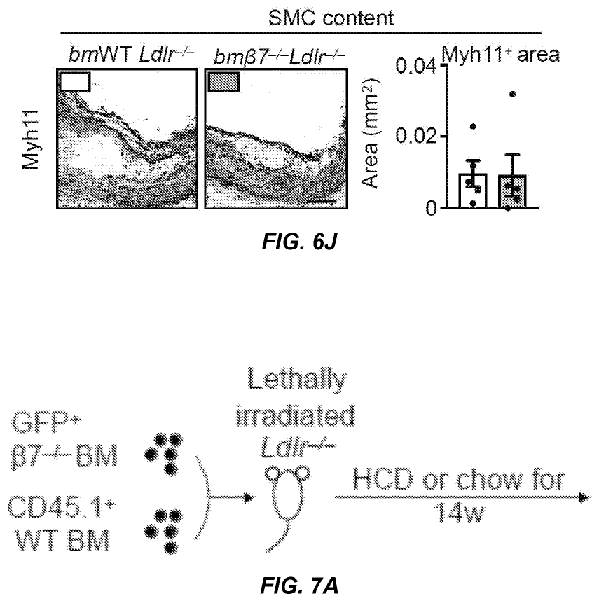

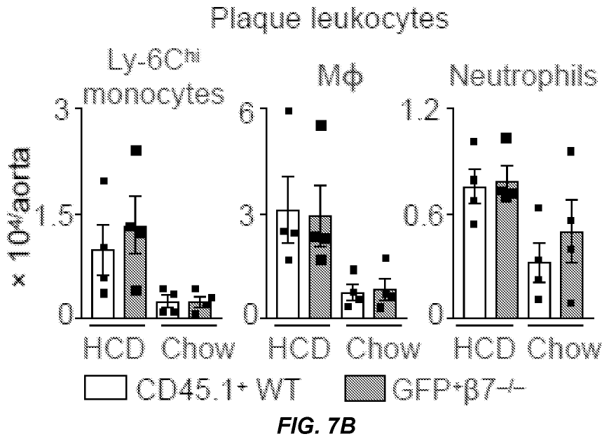

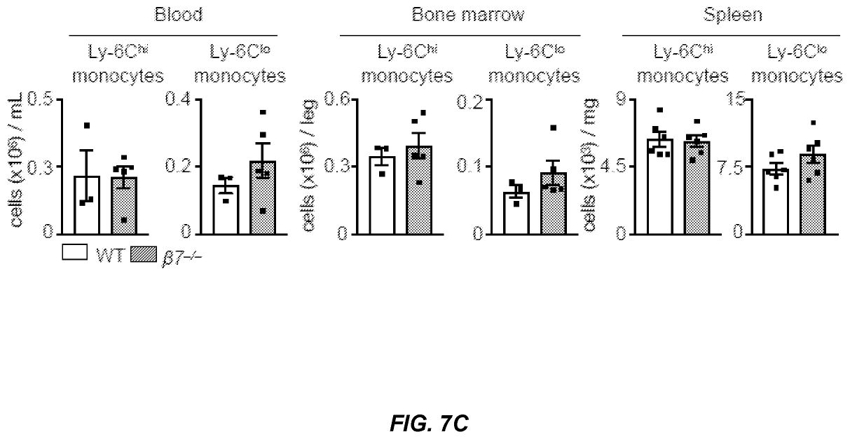

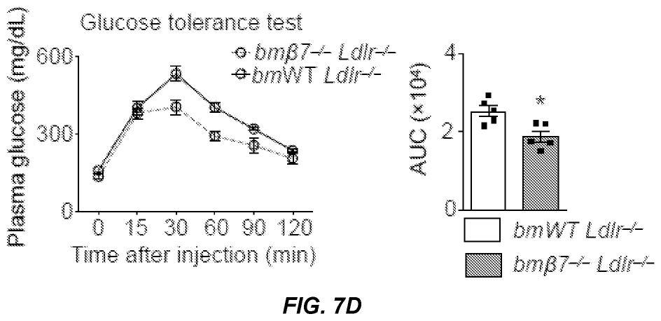

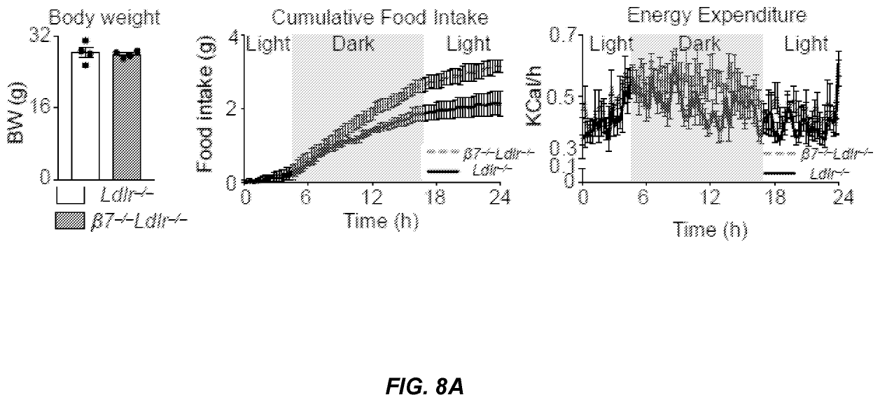

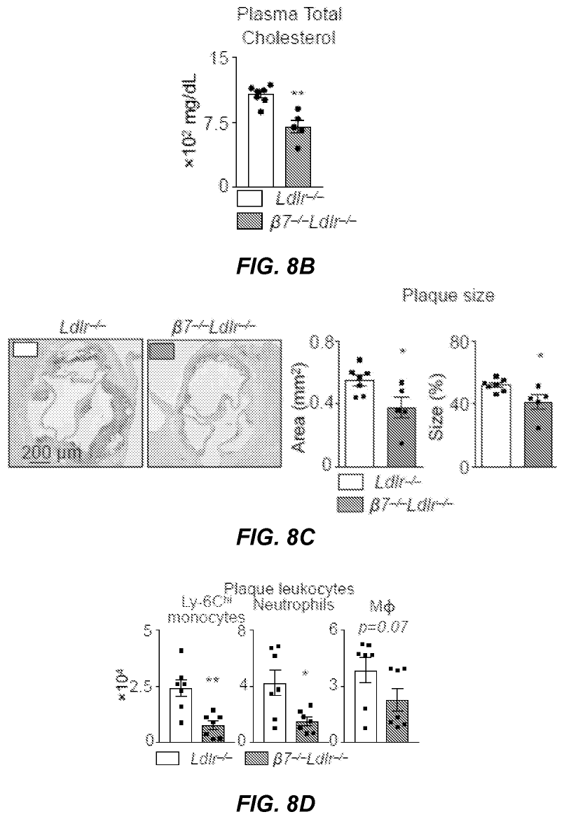

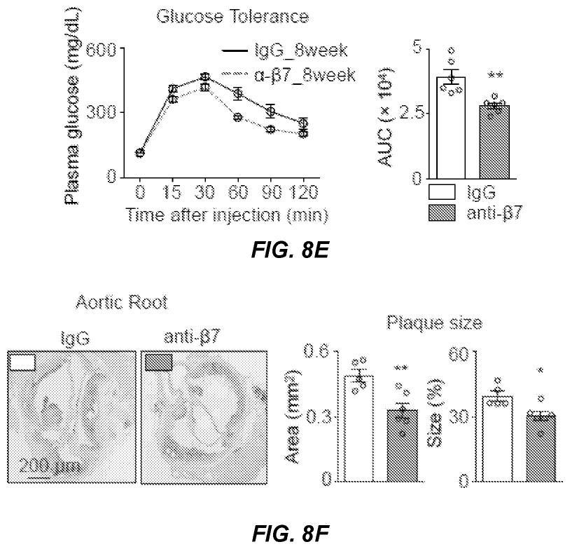

Because β7−/− mice had a higher metabolism and exhibited fewer metabolic syndrome components, we tested whether they had lower rates of atherosclerosis, which is a chronic, lipid-driven inflammatory disease (Swirski, F. K. & Nahrendorf, M. Leukocyte behavior in atherosclerosis, myocardial infarction, and heart failure. Science 339, 161-166 (2013)). We generated Ldlr−/− chimaeras reconstituted with bone marrow from either β7−/− (bmβ7−/−) or wild-type (bmβ7+/+) mice and found that Ldlr−/− mice with β7−/− bone marrow (bmβ7−/−Ldlr−/−) that were fed a diet high in cholesterol (HCD) had considerably lower levels of plasma total cholesterol than controls (bmβ7+/+Ldlr−/−) (FIGS. 3A and 7E). The bmβ7−/−Ldlr−/− mice had lower levels of very-low-density lipoprotein, intermediate-density lipoprotein and low-density lipoprotein, but similar levels of high-density lipoprotein (FIG. 3B). bmβ7−/−Ldlr−/− mice gained weight in a similar manner to bmβ7+/+Ldlr−/− controls (FIG. 6F), yet tended to excrete more cholesterol (FIG. 6G). Moreover, bmβ7−/−Ldlr−/− mice had smaller aortic root lesions (FIG. 3C), with a reduction of around 50% in plaque size and volume (FIGS. 3D. 3E). These changes were driven by differences in the size of the necrotic core (FIGS. 6H-6J) and the number of leukocytes (FIG. 3F), the latter of which was independent of β7-mediated recruitment (FIGS. 7A, 7B). Because hypercholesterolaemia induces leukocytosis (Swirski, F. K. et al. Ly-6Chi monocytes dominate hypercholesterolemia associated monocytosis and give rise to macrophages in atheromata. J. Clin. Invest. 117, 195-205 (2007)), which is a cardiovascular risk factor (Hilgendorf, I. & Swirski, F. K. Making a difference: monocyte heterogeneity in cardiovascular disease. Curr. Atheroscler. Rep. 14, 450-459 (2012)), we also analysed the number of leukocytes in the blood and found fewer circulating Ly-6Chigh and Ly-6Clow monocytes in bmβ7−/−Ldlr−/− mice fed a HCD (FIG. 3G), but not in bmβ7−/−Ldlr−/− mice fed a chow diet (FIG. 7C). Similar to experiments obtained using β7−/− mice, we noted improved glucose tolerance in the bmβ7−/−Ldlr−/− chimaeras (FIG. 7D). We also generated β7−/−Ldlr−/− mice. Following assessment of metabolic functions, which were similar to those in β7−/− mice, we noted that β7−/−Ldlr−/− mice had lower levels of plasma cholesterol, smaller aortic root lesions and fewer aortic leukocytes after HCD (FIGS. 8A-8D). Finally, we injected anti-integrin β7 antibodies into Ldlr−/− mice and found that these mice had improved glucose tolerance and attenuated atherosclerosis (FIGS. 8E-8G). These data show that integrin β7 deficiency protects against atherosclerosis.

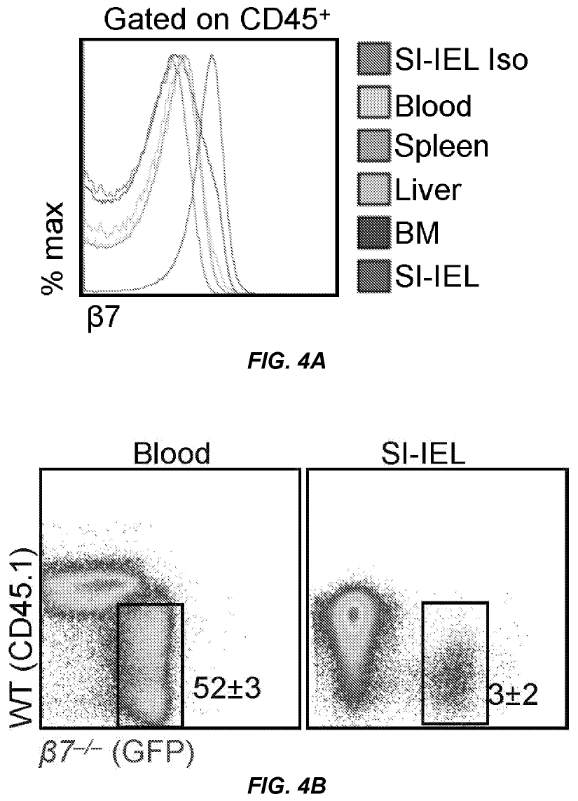

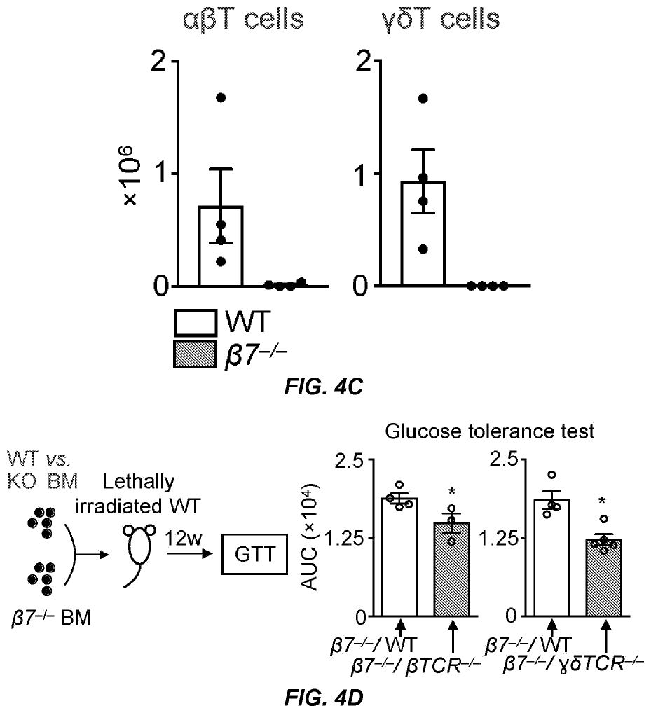

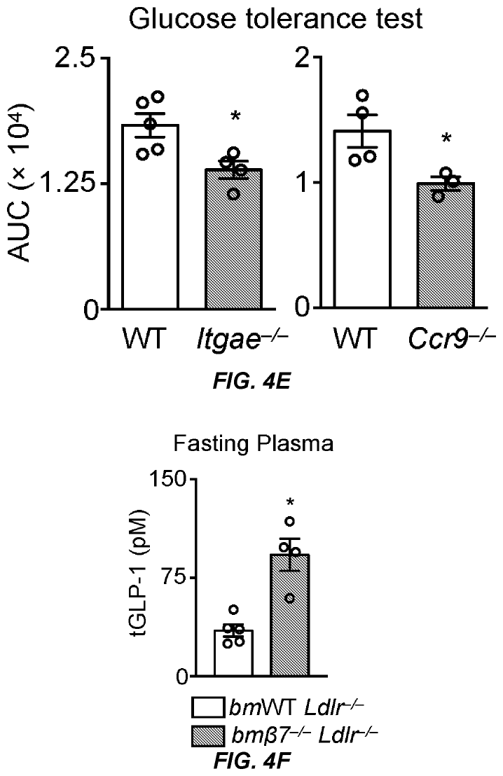

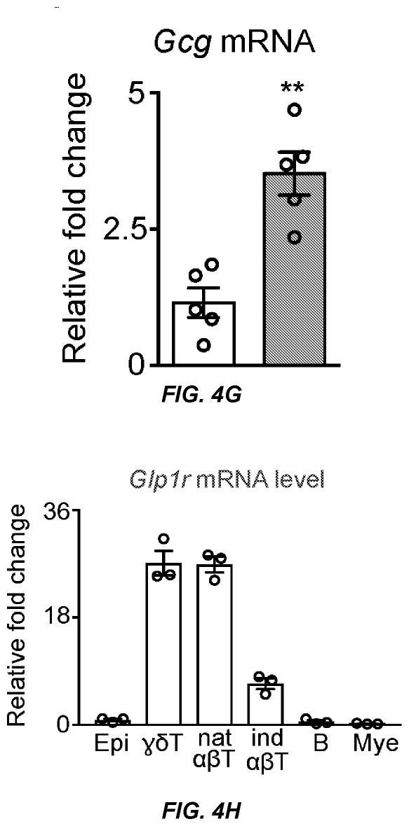

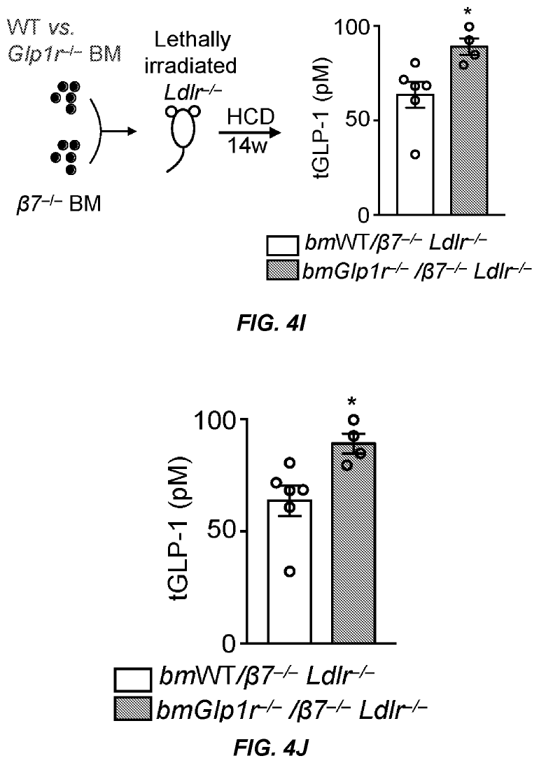

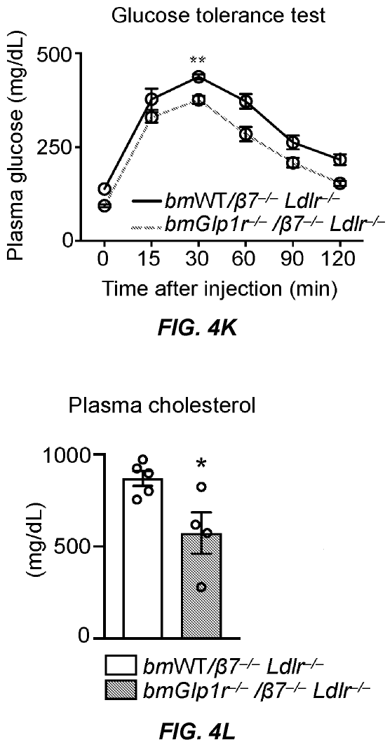

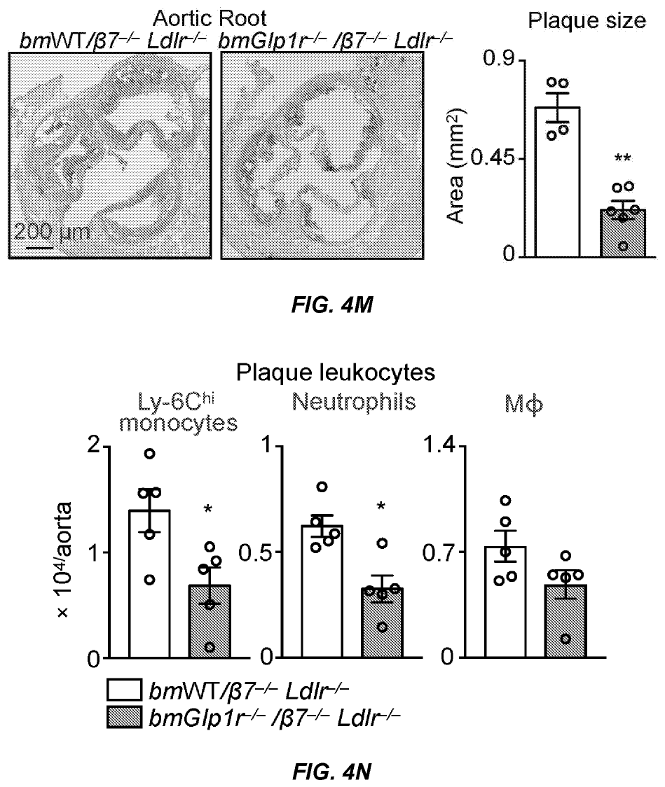

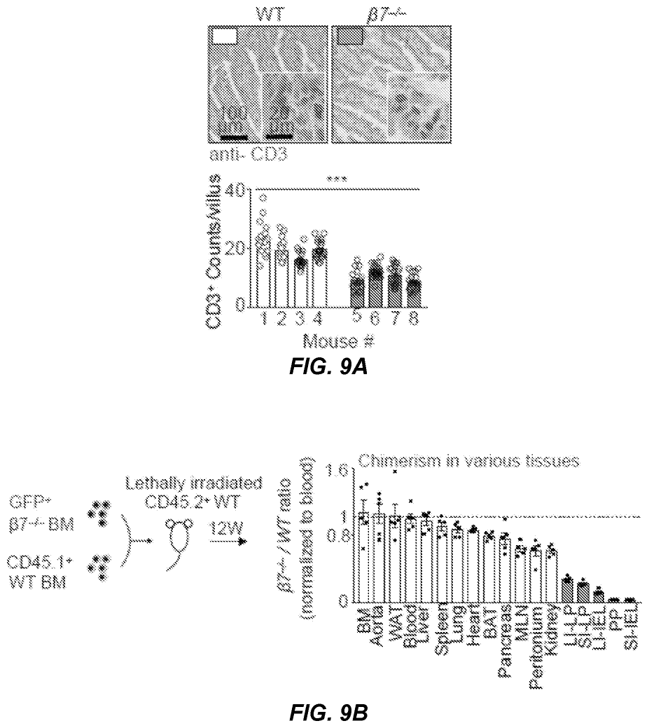

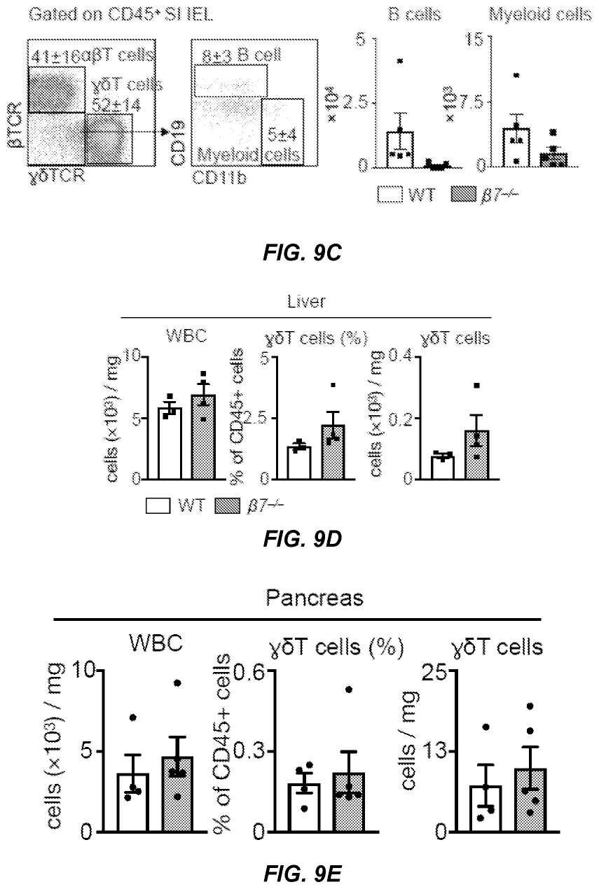

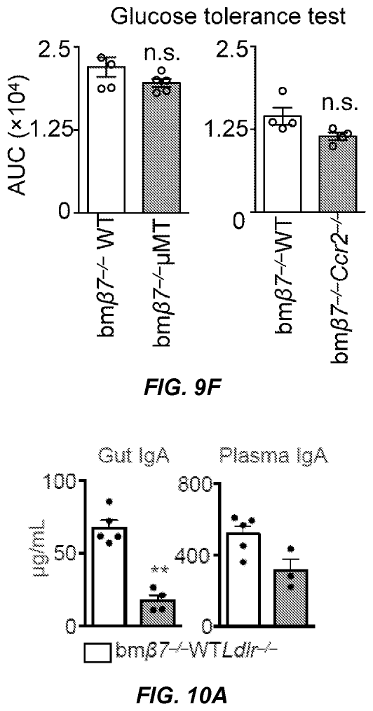

Next, we investigated which cells account for our findings. Intraepithelial lymphocytes that reside in the small intestine had the highest integrin β7 expression (FIG. 4A), in agreement with studies that have shown that integrin β7 guides leukocytes to the gut (Berlin, C. et al. α4β7 integrin mediates lymphocyte binding to the mucosal vascular addressin MAdCAM-1. Cell 74, 185-195 (1993); Wagner, N. et al. Critical role for β7 integrins in formation of the gut-associated lymphoid tissue. Nature 382, 366-370 (1996)). Although the intestinal intraepithelium had fewer CD3+ cells in β7−/− mice (FIG. 9A), we nevertheless analysed the relative ability of β7+ cells to enter tissues (FIG. 9B). The blood contained β7−/− and wild-type cells in similar proportions (FIG. 4B) and—although many tissues accumulated β7−/− and wild-type cells in similar, albeit varied, proportions—considerably fewer β7−/− cells accumulated in the gut and particularly in the small intestine intraepithelium (FIG. 4B and FIG. 9B). These findings therefore confirm that integrin β7 directs leukocytes to the gut.

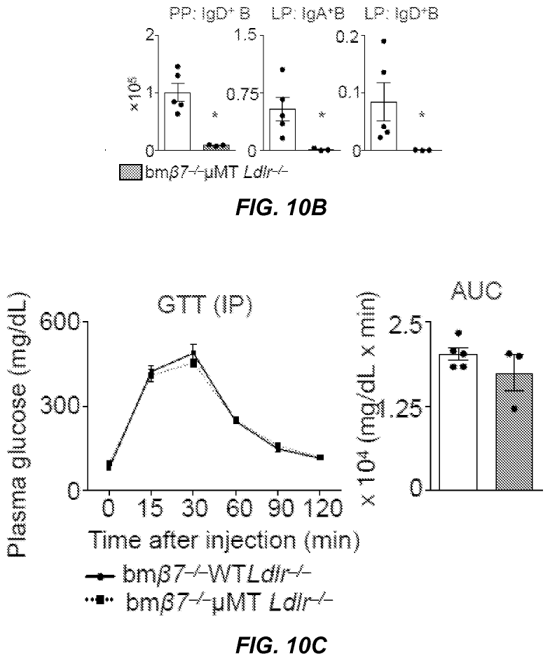

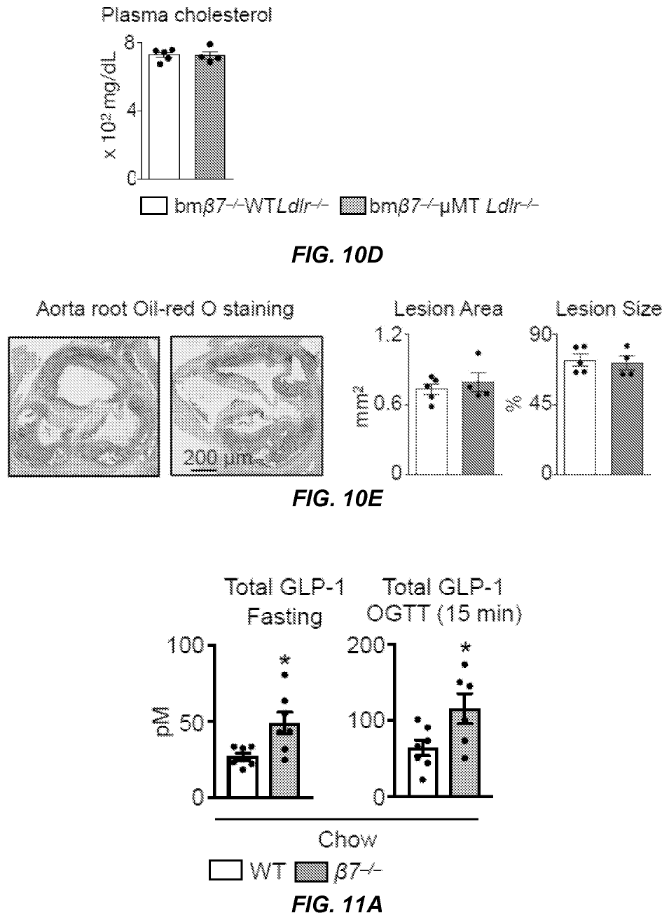

Our results show that the leukocytes that rely on integrin β7 influx to the gut are αβ and γδ T cells (FIG. 4C), B cells and myeloid cells (FIG. 9C). Notably, β7−/− mice had similar numbers of leukocytes as wild-type mice in metabolically important organs such as the liver and pancreas (FIGS. 9D, 9E). Although T cells were the most numerous integrin β7-dependent population that was assessed in the gut, we nevertheless tested which of the three populations (αβ and γδ T cells, B cells or myeloid cells) mediated the metabolic effects. We therefore generated five different mixed chimeric groups of mice (β7−/− wild-type, β7−/−Tcrb−/− (hereafter β7−/−βTCR−/−), β7−/−Tcrd−/− (hereafter β7−/−γδTCR−/−), β7−/−Ighmtm1Cgn (hereafter β7−/−μMT) and β7−/−Ccr2−/−) on a wild-type background and performed a glucose-tolerance test to screen for the metabolic phenotype. We found that specific absence of integrin β7 on either αβ (β7−/−βTCR−/−) or γδ (β7−/−γδTCR−/−) T cells improved glucose tolerance (FIG. 4D), whereas no changes were found in the other mixed chimaeras (FIG. 9F). Both Itgae−/− and Ccr9−/− mice (Schön, M. P. et al. Mucosal T lymphocyte numbers are selectively reduced in integrin αE (CD103)-deficient mice. J. Immunol. 162, 6641-6649 (1999); Uehara, S., Grinberg, A., Farber, J. M. & Love, P. E. A role for CCR9 in T lymphocyte development and migration. J. Immunol. 168, 2811-2819 (2002)) showed a similar improvement in glucose tolerance (FIG. 4E). Moreover, given the importance of B cells for gut homeostasis (Lycke, N. Y. & Bemark, M. The regulation of gut mucosal IgA B-cell responses: recent developments. Mucosal Immunol. 10, 1361-1374 (2017); Fagarasan, S. & Honjo, T. Intestinal IgA synthesis: regulation of front-line body defences. Nat. Rev. Immunol. 3, 63-72 (2003)), we analysed the contribution of this lymphocyte population in more detail, but found no differences in glucose tolerance, cholesterolaemia or atherosclerosis (FIG. 10), indicating that β7-dependent B cells do not contribute to the metabolic phenotype. These data show that intraepithelial αβ and γδ T cells regulate systemic metabolism.

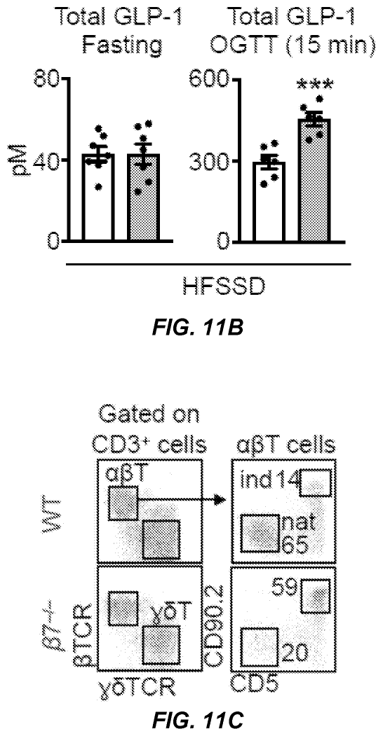

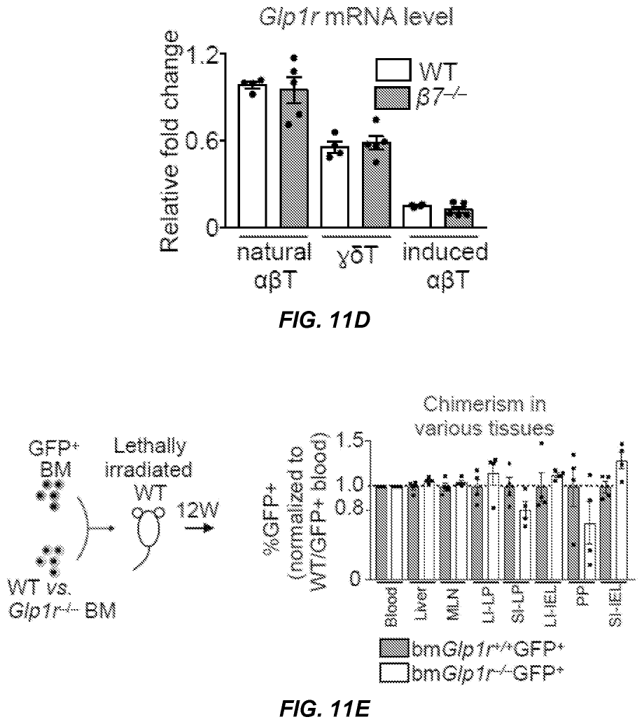

In response to dietary nutrients, enteroendocrine L-cells in the gut produce the incretin hormone GLP-1, which induces postprandial pancreatic insulin secretion and exerts glucose control (Baggio, L. L. & Drucker, D. J. Biology of incretins: GLP-1 and GIP. Gastroenterology 132, 2131-2157 (2007); Kahles, F. et al. GLP-1 secretion is increased by inflammatory stimuli in an IL-6-dependent manner, leading to hyperinsulinemia and blood glucose lowering. Diabetes 63, 3221-3229 (2014)). GLP-1 mediates various other beneficial effects on metabolism, while its analogue improves cardiovascular outcomes in patients with diabetes (Marso, S. P. et al. Liraglutide and cardiovascular outcomes in type 2 diabetes. N. Engl. J. Med. 375, 311-322 (2016); Marso, S. P. et al. Semaglutide and cardiovascular outcomes in patients with type 2 diabetes. N. Engl. J. Med. 375, 1834-1844 (2016)). We found that bmβ7−/−Ldlr−/− mice that were fed a HCD had higher levels of fasting GLP-1 in the plasma (FIG. 4F), along with increased gut Gcg mRNA levels (FIG. 4G). In addition, β7−/− mice had increased levels of GLP-1 compared to wild-type mice that were fed chow or a HFSSD (FIGS. 11A, 11B). To test whether αβ and γδ T cells controlled the bioavailability of GLP-1, we measured the expression of the GLP-1 receptor in gut leukocytes and found that natural αβ and γδ T cells in wild-type mice showed abundant expression of the GLP-1 receptor, consistent with data from Immgen (immgen.org/) and a previous study (Yusta, B. et al. GLP-1R agonists modulate enteric immune responses through the intestinal intraepithelial lymphocyte GLP-1R. Diabetes 64, 2537-2549 (2015)) (FIG. 4H). By contrast, the guts of β7−/− mice were relatively deficient in expression of the GLP-1 receptor, containing fewer and mostly Glp1r^{low} T cells (FIGS. 9A, 11C, 11D). These results indicate that loss of Glp1r^{high} natural IELs is associated with increased plasma levels of the ligand (GLP-1), an observation that is consistent with previous studies that show increased levels of GLP-1 in Glp1r−/− mice (Lamont, B. J. et al. Pancreatic GLP-1 receptor activation is sufficient for incretin control of glucose metabolism in mice. J. Clin. Invest. 122, 388-402 (2012)).

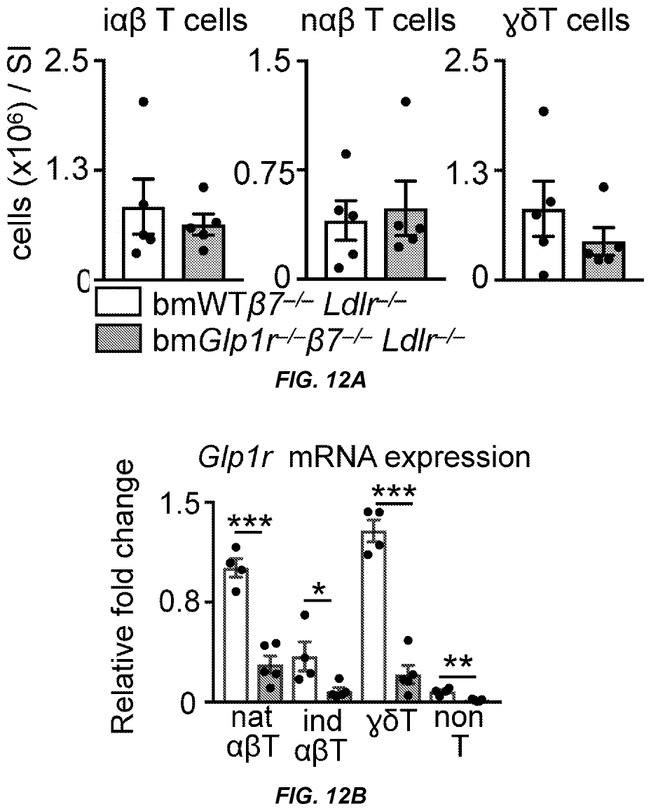

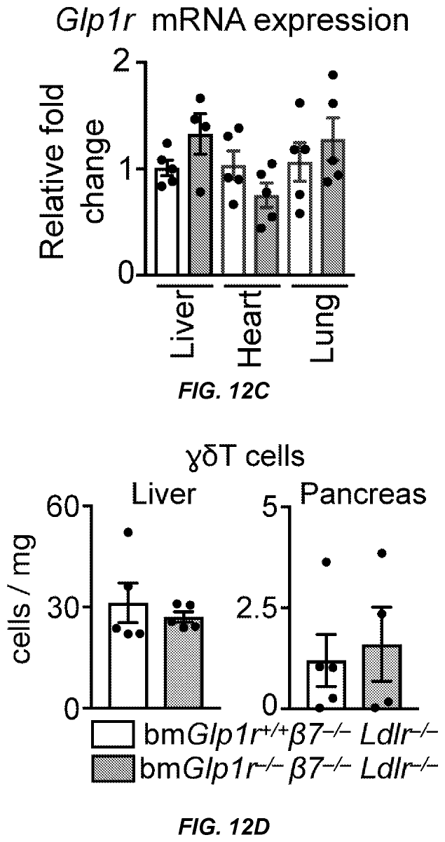

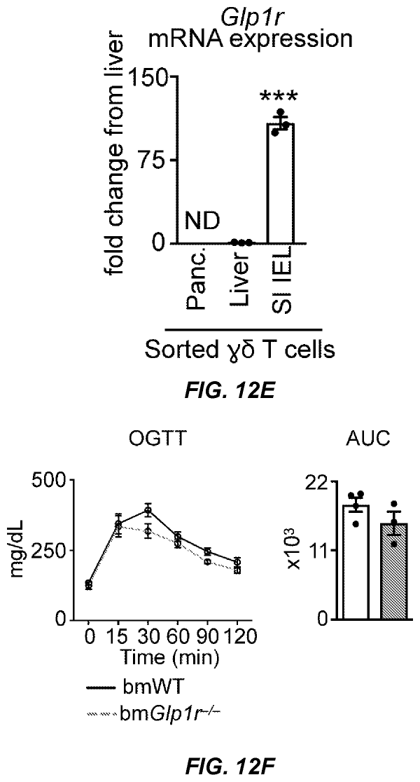

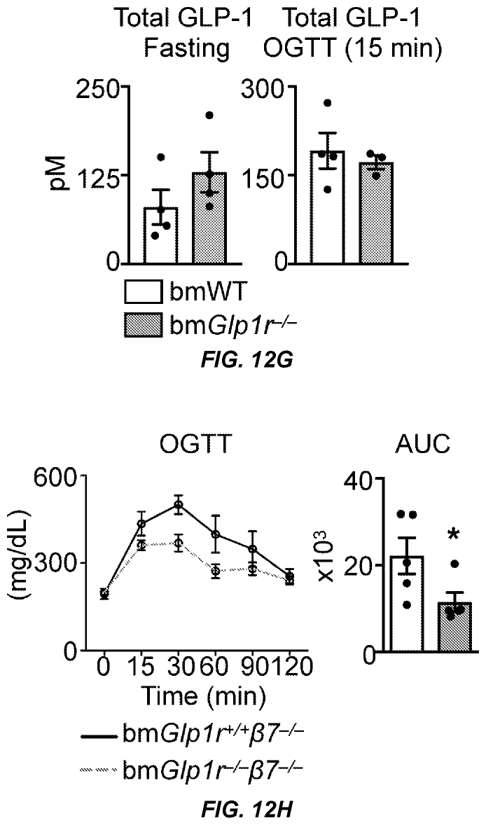

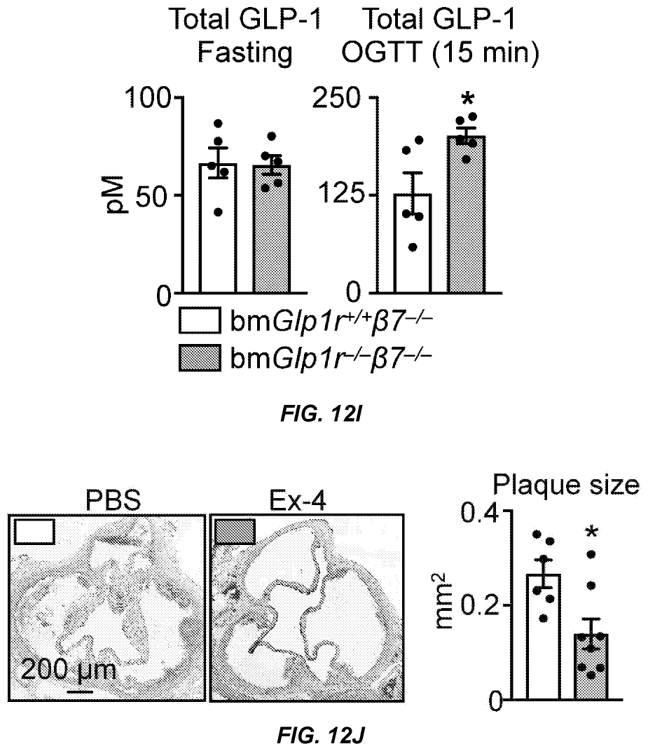

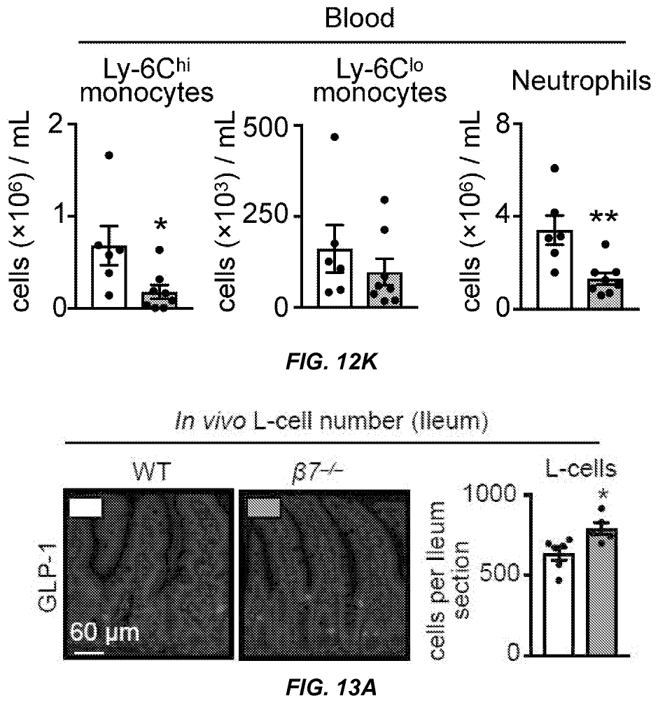

To determine whether loss of the GLP-1 receptor on IELs protects against atherosclerosis through increased systemic levels of GLP-1, we generated mixed chimaeras (bmGlp1r−/−β7−/−; FIG. 4I) on a Ldlr−/− background. After performing quality-control experiments (FIGS. 11E, 12A-12E), we found that bmGlp1r−/−β7−/−Ldlr−/− mice had increased concentrations of GLP-1 (FIG. 4J), were more glucose tolerant (FIG. 4K), less hypercholesterolaemic (FIG. 4L) and developed smaller atherosclerotic lesions (FIG. 4M) with fewer aortic leukocytes (FIG. 4N). Of note, no differences in glucose tolerance and GLP-1 levels were apparent between Glp1r−/− and wild-type chimaeras on a wild-type (that is, not Ldlr−/−) background, although we did see differences in the Glp1r−/−β7−/− mixed chimaeras (FIGS. 12F-12I). By contrast, we found attenuated atherosclerosis in Ldlr−/− mice that received the GLP-1 receptor agonist exendin-4 (FIGS. 12J, 12K), consistent with previous data generated in Apoe−/− mice (Arakawa, M. et al. Inhibition of monocyte adhesion to endothelial cells and attenuation of atherosclerotic lesion by a glucagon-like peptide-1 receptor agonist, exendin-4. Diabetes 59, 1030-1037 (2010); Nagashima, M. et al. Native incretins prevent the development of atherosclerotic lesions in apolipoprotein E knockout mice. Diabetologia 54, 2649-2659 (2011)).

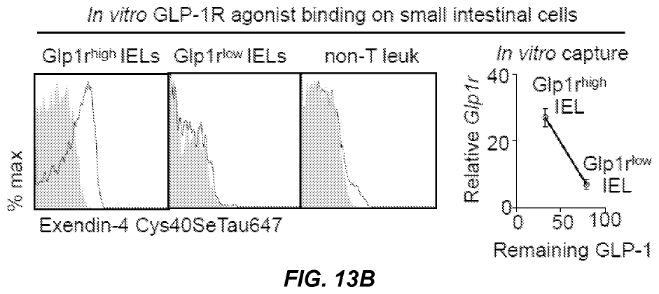

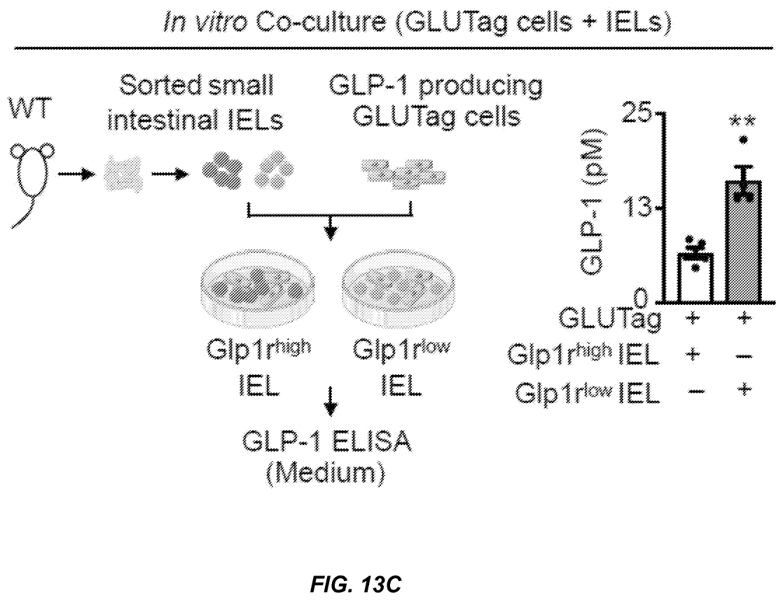

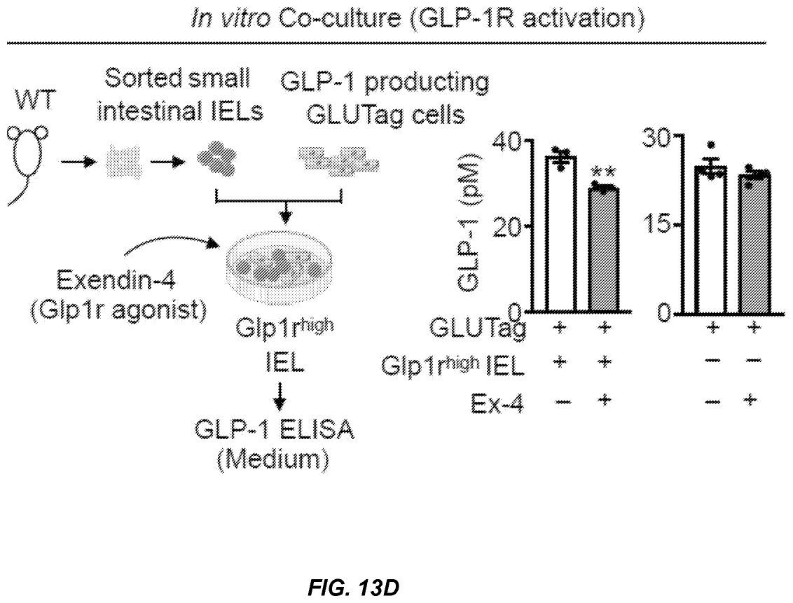

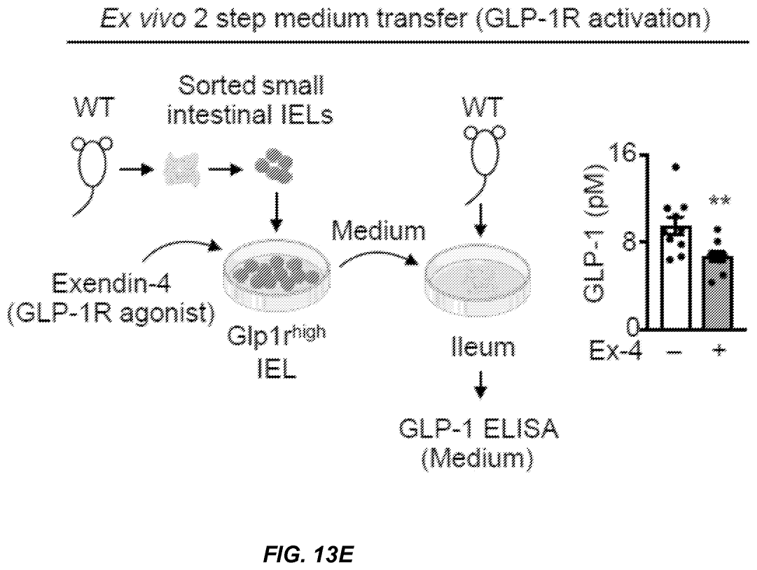

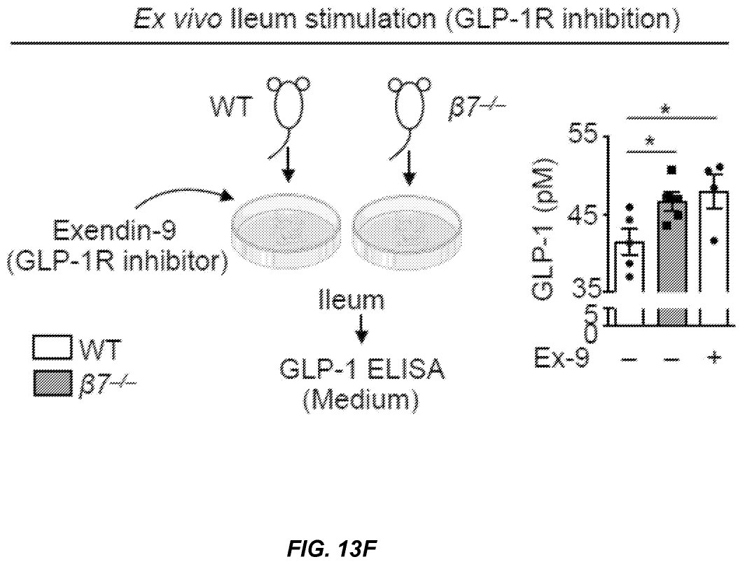

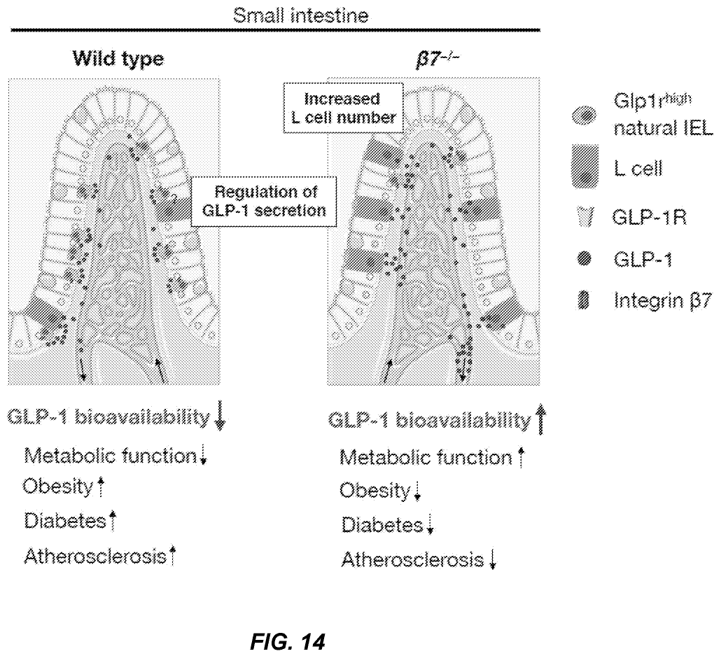

Finally, we reasoned that Glp1r^{high} IELs may be limiting the bioavailability of GLP-1 by several non-mutually exclusive mechanisms. First, we found that β7−/− mice had an increased number of GLP-1-producing L-cells, suggesting that Glp1r^{high} IELs may be controlling GLP-1 production (FIG. 13A). Second, we confirmed that the GLP-1 receptor in Glp1r^{high} IELs is functional, can bind to, capture (FIG. 13B) and control the bioavailability of GLP-1 (FIGS. 13C-13F). These results indicate that the loss of the GLP-1 receptor in IELs triggers a systemic response and limits development of cardiovascular disease through regulation of GLP-1 availability (FIG. 14).

In the gut, T cells help to maintain barrier integrity via various pleiotropic functions. Here we identified β7+ IELs as critical gatekeepers of dietary metabolism. Although the evolution of regulatory mechanisms that sense nutrient availability and regulate energy disposal and storage should offer a survival advantage over organisms that indiscriminately metabolize and expend their ingested energy, such mechanisms may have adverse effects if they become too dominant.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of reducing risk of developing metabolic syndrome or a disease associated with metabolic syndrome in a subject with a family history of metabolic syndrome or a disease associated with metabolic syndrome, elevated blood pressure, dysglycemia, or abdominal obesity, the method comprising administering a therapeutically effective amount of a β7 integrin inhibitor to the subject in need thereof.

2. The method of claim 1, wherein the metabolic syndrome or the disease associated with metabolic syndrome is atherosclerosis.

3. The method of claim 1, wherein the β7 integrin inhibitor is natalizumab or vedolizumab or etrolizumab.

4. The method of claim 1, wherein the therapeutically effective amount of the β7 integrin inhibitor is sufficient to inhibit intraepithelial lymphocyte recruitment to small intestine.

5. The method of claim 4, wherein the β7 integrin inhibitor is delivered directly to the small intestine of the subject, e.g., intraperitoneally, subcutaneously, or by administering the β7 integrin inhibitor in an oral form that remains intact in the stomach but releases the inhibitor once in the small intestine.

6. The method of claim 1, wherein the subject has not been diagnosed with a metabolic syndrome or the disease associated with metabolic syndrome.

7. The method of claim 1, wherein the subject does not have a chronic inflammatory bowel disease.

8. The method of claim 7, wherein the chronic inflammatory bowel disease is irritable bowel syndrome, Crohn's disease, or ulcerative colitis.

9. A method of treating a subject who has been diagnosed with metabolic syndrome or a disease associated with metabolic syndrome, the method comprising administering a therapeutically effective amount of a β7 integrin inhibitor to the subject in need thereof.

10. The method of claim 9, wherein the metabolic syndrome or the disease associated with metabolic syndrome is atherosclerosis.

11. The method of claim 9, wherein the β7 integrin inhibitor is natalizumab or vedolizumab or etrolizumab.

12. The method of claim 9, wherein the therapeutically effective amount of the β7 integrin inhibitor is sufficient to inhibit intraepithelial lymphocyte recruitment to small intestine.

13. The method of claim 12, wherein the β7 integrin inhibitor is delivered directly to the small intestine of the subject, e.g., intraperitoneally, subcutaneously, or by administering the β7 integrin inhibitor in an oral form that remains intact in the stomach but releases the inhibitor once in the small intestine.

14. The method of claim 9, wherein the subject does not have a chronic inflammatory bowel disease.

15. The method of claim 14, wherein the chronic inflammatory bowel disease is irritable bowel syndrome, Crohn's disease, or ulcerative colitis.

16. A method of reducing risk of developing metabolic syndrome or a disease associated with metabolic syndrome in a subject with family history of metabolic syndrome or a disease associated with metabolic syndrome, elevated blood pressure, dysglycemia, or abdominal obesity, the method comprising administering a therapeutically effective amount of a CCR9 inhibitor or a GLP-1 agonist to the subject in need thereof.

17. The method of claim 16, wherein the metabolic syndrome or a disease associated with metabolic syndrome is atherosclerosis.

18. The method of claim 16, wherein the CCR9 inhibitor is CCX025 or CCX282.

19. The method of claim 16, wherein the subject has not been diagnosed with a metabolic syndrome or a disease associated with metabolic syndrome.

20. The method of claim 16, wherein the subject does not have a chronic inflammatory bowel disease.

21. The method of claim 19, wherein the chronic inflammatory bowel disease is irritable bowel syndrome, Crohn's disease, or ulcerative colitis.
